SEQUENCE LISTING (107 sequences)

SEQ ID NO: 1 - protein, 9 aa, Homo Sapiens
SLDKFLASV

SEQ ID NO: 2 - protein, 10 aa, Homo Sapiens
IGDEAIEKPT

SEQ ID NO: 3 - protein, 11 aa, Homo Sapiens
VADALTNAVAH

SEQ ID NO: 4 - protein, 11 aa, Homo Sapiens
LELAGNAARDN

SEQ ID NO: 5 - protein, 10 aa, Homo Sapiens
LVNEVTEFAK

SEQ ID NO: 6 - protein, 10 aa, Homo Sapiens
DLLDDLKSEL

SEQ ID NO: 7 - protein, 10 aa, Homo Sapiens
WLQGSQELPR

SEQ ID NO: 8 - protein, 11 aa, Homo Sapiens
IKDPDASKPED

SEQ ID NO: 9 - protein, 12 aa, Homo Sapiens
DLKSVLGQLGIT

SEQ ID NO: 10 - protein, 13 aa, Homo Sapiens
GKVNVDEVGGEAL

SEQ ID NO: 11 - protein, 13 aa, Homo Sapiens
VNVDEVGGEALGR

SEQ ID NO: 12 - protein, 12 aa, Homo Sapiens
FSDGLAHLDNLK

SEQ ID NO: 13 - protein, 13 aa, Homo Sapiens
KVVATTQMQAADA

SEQ ID NO: 14 - protein, 13 aa, Homo Sapiens
  MOD_RES (6)..(6): Xaa = oxidized methionine
SGGTTXYPGIADR

SEQ ID NO: 15 - protein, 11 aa, Homo Sapiens
FYAPELLFFAK

SEQ ID NO: 16 - protein, 12 aa, Homo Sapiens
FQNSAILTIQPK

SEQ ID NO: 17 - protein, 13 aa, Homo Sapiens
LTEPADTITDAVK

SEQ ID NO: 18 - protein, 14 aa, Homo Sapiens
LVTEVENGGSLGSK

SEQ ID NO: 19 - protein, 12 aa, Homo Sapiens
  MOD_RES (12)..(12): Xaa = oxidized methionine
DLAGRDLTDYLX

SEQ ID NO: 20 - protein, 13 aa, Homo Sapiens
LTGLLDLALEKDY

SEQ ID NO: 21 - protein, 14 aa, Homo Sapiens
TNAVAHVDDMPNAL

SEQ ID NO: 22 - protein, 14 aa, Homo Sapiens
DGLAHLDNLKGTFA

SEQ ID NO: 23 - protein, 14 aa, Homo Sapiens
SLDKFLASVSTVLT

SEQ ID NO: 24 - protein, 15 aa, Homo Sapiens
VGAHAGEYGAEALER

SEQ ID NO: 25 - protein, 16 aa, Homo Sapiens
RPSGNLVSVLSGAEGS

SEQ ID NO: 26 - protein, 15 aa, Homo Sapiens
VNVDEVGGEALGRLL

SEQ ID NO: 27 - protein, 15 aa, Homo Sapiens
SDGLAHLDNLKGTFA

SEQ ID NO: 28 - protein, 15 aa, Homo Sapiens
VDDMPNALSALSDLH

SEQ ID NO: 29 - protein, 16 aa, Homo Sapiens
GAFSDGLAHLDNLKGT

SEQ ID NO: 30 - protein, 15 aa, Homo Sapiens
SNKYDPPLEDGAMPS

SEQ ID NO: 31 - protein, 14 aa, Homo Sapiens
MLLADQGQSWKEEV

SEQ ID NO: 32 - protein, 13 aa, Homo Sapiens
AVHYLDETEQWEK

SEQ ID NO: 33 - protein, 16 aa, Homo Sapiens
VGAHAGEYGAEALERM

SEQ ID NO: 34 - protein, 16 aa, Homo Sapiens
  MOD_RES (6)..(6): Xaa = oxidized methionine
AHVDDXPNALSALSDL

SEQ ID NO: 35 - protein, 16 aa, Homo Sapiens
IAPQLSTEELVSLGEK

SEQ ID NO: 36 - protein, 17 aa, Homo Sapiens
ALWGKVNVDEVGGEALG

SEQ ID NO: 37 - protein, 15 aa, Homo Sapiens
YAASSYLSLTPEQWK

SEQ ID NO: 38 - protein, 16 aa, Homo Sapiens
VANPSGNLTETYVQDR

SEQ ID NO: 39 - protein, 16 aa, Homo Sapiens
APVPTGEVYFADSFDR

SEQ ID NO: 40 - protein, 18 aa, Homo Sapiens
TALWGKVNVDEVGGEALG

SEQ ID NO: 41 - protein, 18 aa, Homo Sapiens
SAVTALWGKVNVDEVGGE

SEQ ID NO: 42 - protein, 18 aa, Homo Sapiens
  MOD_RES (14)..(14): Xaa = oxidized methionine
FESFGDLSTPDAVXGNPK

SEQ ID NO: 43 - protein, 20 aa, Homo Sapiens
SLDMDSIIAEVKAQYEDIAN

SEQ ID NO: 44 - protein, 8 aa, Homo Sapiens
LSALSDLH

SEQ ID NO: 45 - protein, 9 aa, Homo Sapiens
GEALGRLLV

SEQ ID NO: 46 - protein, 11 aa, Homo Sapiens
TPDAVMGNPKV

SEQ ID NO: 47 - protein, 11 aa, Homo Sapiens
SASNMAIVDVK

SEQ ID NO: 48 - protein, 10 aa, Homo Sapiens
ILDVLEEIPK

SEQ ID NO: 49 - protein, 12 aa, Homo Sapiens
TEVENGGSLGSK

SEQ ID NO: 50 - protein, 13 aa, Homo Sapiens
NVDEVGGEALGRL

SEQ ID NO: 51 - protein, 12 aa, Homo Sapiens
ISKQEYDESGPS

SEQ ID NO: 52 - protein, 11 aa, Homo Sapiens
EYVQLISVYEK

SEQ ID NO: 53 - protein, 13 aa, Homo Sapiens
KVPQVSTPTLVEV

SEQ ID NO: 54 - protein, 13 aa, Homo Sapiens
AEFAEVSKLVTDL

SEQ ID NO: 55 - protein, 13 aa, Homo Sapiens
FFESFGDLSTPDA

SEQ ID NO: 56 - protein, 14 aa, Homo Sapiens
  MOD_RES (3)..(3): Xaa = oxidized methionine
DDXPNALSALSDLH

SEQ ID NO: 57 - protein, 15 aa, Homo Sapiens
LAKVDATEESDLAQQ

SEQ ID NO: 58 - protein, 15 aa, Homo Sapiens
  MOD_RES (2)..(2): Xaa = oxidized methionine
IXDPNIVGSEHYDVA

SEQ ID NO: 59 - protein, 15 aa, Homo Sapiens
SDPEQGVEVTGQYER

SEQ ID NO: 60 - protein, 17 aa, Homo Sapiens
GAFSDGLAHLDNLKGTF

SEQ ID NO: 61 - protein, 13 aa, Homo Sapiens
LGPHAGDVEGHLS

SEQ ID NO: 62 - protein, 33 aa, Homo Sapiens
DGVSGGEGKGGSDGGGSHRKEGEEADAPGVIPG

SEQ ID NO: 63 - protein, 9 aa, Homo Sapiens
DFDDFNLED

SEQ ID NO: 64 - protein, 17 aa, Homo Sapiens
  MOD_RES (2)..(2): Xaa = hydroxylated proline
EXGSXGENGAXGQMGPR

SEQ ID NO: 65 - protein, 23 aa, Homo Sapiens
  MOD_RES (6)..(6): Xaa = hydroxylated proline
GNSGEXGAXGSKGDTGAKGEPGX

SEQ ID NO: 66 - protein, 30 aa, Homo Sapiens
  MOD_RES (7)..(7): Xaa = hydroxylated proline
ERGSPGXAGPKGSXGEAGRXGEAGLXGAKG

SEQ ID NO: 67 - protein, 18 aa, Homo Sapiens
  MOD_RES (2)..(2): Xaa = hydroxylated proline
SXGSPGPDGKTGPXGPAG

SEQ ID NO: 68 - protein, 23 aa, Homo Sapiens
  MOD_RES (6)..(6): Xaa = hydroxylated proline
DGKTGXPGPAGQDGRPGPXGXXG

SEQ ID NO: 69 - protein, 13 aa, Homo Sapiens
  MOD_RES (10)..(10): Xaa = hydroxylated proline
DGEAGAQGPXGPA

SEQ ID NO: 70 - protein, 14 aa, Homo Sapiens
  MOD_RES (1)..(1): Xaa = hydroxylated proline
XPGEAGKXGEQGVP

SEQ ID NO: 71 - protein, 12 aa, Homo Sapiens
  MOD_RES (2)..(2): Xaa = hydroxylated proline
KXGEQGVXGDLG

SEQ ID NO: 72 - protein, 16 aa, Homo Sapiens
  MOD_RES (4)..(4): Xaa = hydroxylated proline
DGQXGAKGEXGDAGAK

SEQ ID NO: 73 - protein, 13 aa, Homo Sapiens
  MOD_RES (2)..(2): Xaa = hydroxylated proline
RXGEVGPXGPXGP

SEQ ID NO: 74 - protein, 13 aa, Homo Sapiens
  MOD_RES (3)..(3): Xaa = hydroxylated proline
GPXGESGREGAXG

SEQ ID NO: 75 - protein, 32 aa, Homo Sapiens
  MOD_RES (8)..(8): Xaa = hydroxylated proline
ESGREGAXGAEGSXGRDGSXGAKGDRGETGPA

SEQ ID NO: 76 - protein, 22 aa, Homo Sapiens
  MOD_RES (4)..(4): Xaa = hydroxylated proline
EGSXGRDGSXGAKGDRGETGPA

SEQ ID NO: 77 - protein, 20 aa, Homo Sapiens
  MOD_RES (10)..(10): Xaa = hydroxylated proline
DGESGRPGRXGERGLXGPXG

SEQ ID NO: 78 - protein, 20 aa, Homo Sapiens
  MOD_RES (7)..(7): Xaa = hydroxylated proline
DGESGRXGRXGERGLXGPXG

SEQ ID NO: 79 - protein, 15 aa, Homo Sapiens
  MOD_RES (2)..(2): Xaa = hydroxylated proline
SXGGXGSDGKXGPXG

SEQ ID NO: 80 - protein, 14 aa, Homo Sapiens
  MOD_RES (12)..(12): Xaa = hydroxylated proline
GPGGDKGDTGPXGP

SEQ ID NO: 81 - protein, 48 aa, Homo Sapiens
  MOD_RES (5)..(5): Xaa = hydroxylated proline
LQGLXGTGGXXGENGKXGEXGXKGDAGAPGAPGGKGDAGAPGERGXXG

SEQ ID NO: 82 - protein, 21 aa, Homo Sapiens
  MOD_RES (2)..(2): Xaa = hydroxylated proline
AXGAPGGKGDAGAXGERGPXG

SEQ ID NO: 83 - protein, 18 aa, Homo Sapiens
  MOD_RES (2)..(2): Xaa = hydroxylated proline
AXGPQGPRGDXGETGERG

SEQ ID NO: 84 - protein, 15 aa, Homo Sapiens
  MOD_RES (7)..(7): Xaa = hydroxylated proline
KGEAGLXGAXGSPGQ

SEQ ID NO: 85 - protein, 22 aa, Homo Sapiens
  MOD_RES (3)..(3): Xaa = hydroxylated proline
GPXGKAGEDGHXGKPGRXGERG

SEQ ID NO: 86 - protein, 30 aa, Homo Sapiens
  MOD_RES (2)..(2): Xaa = hydroxylated lysine
LXGQXGAXGVKGEXGAXGENGTPGQTGARG

SEQ ID NO: 87 - protein, 30 aa, Homo Sapiens
  MOD_RES (2)..(2): Xaa = hydroxylated lysine
LXGQXGAXGVXGEXGAXGENGTXGQTGARG

SEQ ID NO: 88 - protein, 29 aa, Homo Sapiens
  MOD_RES (1)..(1): Xaa = hydroxylated lysine
XGQXGAXGVXGEXGAXGENGTXGQTGARG

SEQ ID NO: 89 - protein, 18 aa, Homo Sapiens
  MOD_RES (2)..(2): Xaa = hydroxylated proline
SXGNIGPAGKEGPVGLXG

SEQ ID NO: 90 - protein, 20 aa, Homo Sapiens
DEAGSEADHEGTHSTKRGHA

SEQ ID NO: 91 - protein, 22 aa, Homo Sapiens
DEAGSEADHEGTHSTKRGHAKS

SEQ ID NO: 92 - protein, 24 aa, Homo Sapiens
DEAGSEADHEGTHSTKRGHAKSRP

SEQ ID NO: 93 - protein, 10 aa, Homo Sapiens
ATKTVGSDTF

SEQ ID NO: 94 - protein, 15 aa, Homo Sapiens
SGDSDDDEPPPLPRL

SEQ ID NO: 95 - protein, 26 aa, Homo Sapiens
IPVKQADSGSSEEKQLYNKYPDAVAT

SEQ ID NO: 96 - protein, 9 aa, Homo Sapiens
GEYKFQNAL

SEQ ID NO: 97 - protein, 17 aa, Homo Sapiens
TGLSMDGGGSPKGDVDP

SEQ ID NO: 98 - protein, 16 aa, Homo Sapiens
GLSMDGGGSPKGDVDP

SEQ ID NO: 99 - protein, 9 aa, Homo Sapiens
SGSVIDQSR

SEQ ID NO: 100 - protein, 13 aa, Homo Sapiens
DQSRVLNLGPITR

SEQ ID NO: 101 - protein, 18 aa, Homo Sapiens
SGSVIDQSRVLNLGPITR

SEQ ID NO: 102 - protein, 19 aa, Homo Sapiens
SGSVIDQSRVLNLGPITRK

SEQ ID NO: 103 - protein, 16 aa, Homo Sapiens
VGGGEQPPPAPAPRRE

SEQ ID NO: 104 - protein, 15 aa, Homo Sapiens
REVQSKIGYGRQIIS

SEQ ID NO: 105 - protein, 24 aa, Homo Sapiens
ELMTGELPYSHINNRDQIIFMVGR

SEQ ID NO: 106 - protein, 21 aa, Homo Sapiens
TGSLPYSHIGSRDQIIFMVGR

SEQ ID NO: 107 - protein, 18 aa, Homo Sapiens
GIVLYELMTGELPYSHIN

(19) United States
(12) Patent Application Publication
(10) Pub. No.: US 2014/0027283 A1
(43) Pub. Date: Jan. 30, 2014

(54) METHOD AND MARKER FOR THE DIAGNOSIS OF A BILE DUCT STRICTURE AND OF A CHOLANGIOCELLULAR CARCINOMA IN BILE

(71) Applicant: Harald Mischak, Sehnde (DE)

(72) Inventor: Harald Mischak, Sehnde (DE)

(73) Assignee: MOSAIQUES DIAGNOSTICS AND THERAPEUTICS AG, Hannover (DE)

(21) Appl. No.: 13/992,175

(22) PCT Filed: Dec. 12, 2011
(86) PCT No.: PCT/EP2011/072441

§ 371 Date: Oct. 8, 2013

(30) Foreign Application Priority Data
Dec. 10, 2010 (EP) 10194613.5
Jul. 15, 2011 (EP) 11174088.2

(51) Int. Cl. G01N 33/487; G01N 27/447; H01J 49/00

(52) U.S. Cl. CPC G01N 33/487; H01J 49/0027; G01N 27/447
USPC 204/451; 73/61.41; 250/282

(57) ABSTRACT

A method for the diagnosis of a benign or malignant bile duct stricture and/or of a CCC, comprising the step of determining at least three polypeptide markers in a body fluid, wherein the polypeptide marker belongs to those markers which in table 1 and/or table 2 a and/or b are characterized by values for the molecular mass and the migration time.

The present invention relates to the differentiation of a benign or malignant bile duct stricture from choledocholithiasis, and to the differentiation of a cholangiocellular carcinoma from a primary sclerosing cholangitis in the presence of an unclear bile duct stenosis, in the bile.

Bile duct stenoses can have both benign and malignant causes. The occlusion of the bile ducts, among others, by calculi between the donor bile duct and recipient bile duct is considered benign. The most frequent stenosing tumors include the cholangiocellular carcinoma (CCC), pancreatic carcinoma, hepatocellular carcinoma (HCC), but also metastatic spreads of other carcinomas (Khan et al., J Hepatol 2002, Vol. 37, pages 806-813). Clinical, radiological and endoscopic methods are used for diagnosis, but an unambiguous diagnosis is not possible without a biopsy or surgical intervention in many cases (Malhi and Gores, Aliment Pharmacol Ther, 2006, Vol. 23, pages 1287-1296). In particular, the confirmation of an extrahepatic bile duct stenosis is difficult because of the intricate site. Studies showed that even the combination of brush cytologies during endoscopic retrograde cholangiopancreatography (ERCP) and endoscopic biopsies yielded an unambiguous diagnosis only in one third of the cases (Lazaridis and Gores, Gastroenterology, 2005, Vol. 128, pages 1655-1667, Gores, Hepatology, 2003, Vol. 37, pages 961-969).

In particular, the early detection of a CCC has considerable therapeutic consequences. A CCC is based on the malignant degeneration of cholangiocytes throughout the biliary system and is mostly detected only in an advanced stage, so that less than 50% of the patients can undergo surgery and thus can be treated curatively (Malhi and Gores, Aliment Pharmacol Ther, 2006, Vol. 23, pages 1287-1296, Singh and Patel, Curr Opin Gastroenterol, 2006, Vol. 22, pages 294-299). An effective chemotherapy has not been available to date. A critical risk group for CCC has been successfully characterized. These are patients with primary sclerosing cholangitis (PSC), but a satisfactory monitoring strategy for early detection of carcinomas for this group of patients has not been available to date (LaRusso et al., Hepatology, 2006, Vol. 44, pages 746-764). Patients with PSC suffer from a chronic fibrosing cholestatic disease that gradually obstructs the biliary ducts and is associated with ulcerative colitis. In the initial stage, these patients are mostly without complaints, and itching, tiredness and icterus develop only later (LaRusso et al., Hepatology, 2006, Vol. 44, pages 746-764). In the laboratory, increased levels of alkaline phosphatase and gamma-glutamyl transferase are striking first, and also bilirubin later on. Anti-neutrophile cyto-plasmatic antibodies (p-ANCA) are found in about 85% of the patients, but this marker has no high specificity for PSC and is also found in other diseases, such as in patients with chronic inflammatory bowel disease without PSC. Therefore, the diagnosis is still based on imaging the bile ducts with the typical picture of multiple bile duct stenoses. The risk of developing CCC is highly increased in patients with PSC as compared to the normal population: In a large multicenter study from Sweden with 305 PSC patients, a CCC was observed in 8% thereof, and 44% of the patients were without symptoms when the diagnosis was established (Broome et al., Gut, 1996, Vol. 38, pages 610-615). Other studies show significantly higher carcinoma rates (LaRusso et al., Hepatology, 2006, Vol. 44, pages 746-764, West et al., Br J Cancer, 2006, Vol. 94, pages 1751-1758). Thus, a study with 273 PSC patients performed on the Medizinische Hochschule Hannover showed a carcinoma rate of 14% (Tischendorf et al., Am J Gastroenterol, 2007, Vol. 102, pages 107-114). Only an early diagnosis leads to a timely liver transplantation and thus to a curative therapy.

In most patients with PSC, diagnostic or therapeutic ERCP as well as radiological checkups (magnetic resonance cholangiopancreatography, MRCP) are performed on a regular basis for monitoring, but all examinations known to date for early detection of CCC have not been sensitive enough. The determination of tumor markers increases in importance, but these are currently not sufficient either, in particular, for early detection of CCC, rather serving as follow-up parameters. The serum marker CA19-9 is currently the one most often used for diagnosing CCC, but other tumor markers and biomarkers are urgently required for a better sensitivity and specificity (Levy et al., Dig Dis Sci, 2005, Vol. 50, pages 1734-1740, Lempinen et al., J Hepatol, 2007, Vol. 47, pages 677-683).

To conclude, the necessity of a search for suitable parameters for recognizing and confirming an unclear bile duct stenosis exists. A physiologically plausible approach is the examination of the bile from the bile duct in ERCP, since malignant tumors infiltrate the bile ducts and can secrete proteins. Therefore, a protein analysis of the bile in patients with bile duct stenoses when the basic diseases are known is of great importance not only diagnostically, but also in terms of pathophysiology. The examination of the complex protein composition of the bile (also in comparison with other body fluids, such as urine and blood) is a new diagnostic method that defines particular peptide patterns by mass-spectroscopic analyses. The sampling of the bile is effected endoscopically. In principle, it would be desirable for the patients if the diagnosis could be effected also from other body fluids.

Therefore, it is the object of the present invention to provide processes and means for the differentiation of benign and malignant bile duct strictures in general, and of a CCC from a PSC or other benign strictures, such as choledocholithiasis, when an unclear bile duct stenosis has been found, in particular.

This object is achieved by a process for the diagnosis of a benign or malignant bile duct stricture and of a CCC, comprising the step of determining the presence or absence or amplitude of at least three polypeptide markers in a bile sample, wherein said polypeptide markers for the diagnosis of a bile duct stricture or CCC are selected from the markers characterized in Tables 1, 2a and 2b by values for the molecular masses and migration times.

TABLE 1
List of markers that enable the differentiation of benign and malignant bile
duct strictures from choledocholithiasis in a bile sample in a multimarker model.

| | | | | | Stricture | | Choledocholithiasis | |
|---|---|---|---|---|---|---|---|---|
| | | more | Mass | CE time | | mean log amp | | mean log amp |
| Protein ID | preferably | preferably | [Da] | [min] | Frequency | (log median) | Frequency | (log median) |
| 164 | 164 | 164 | 834.47 | 21.99 | 0.26 | 1.54(1.52) | 0.48 | 2.32(2.22) |
| 965 | | | 917.49 | 30.35 | 0.52 | 1.78(1.77) | 0.26 | 2.04(1.82) |
| 988 | 988 | 988 | 919.43 | 29.9 | 0.18 | 1.94(1.76) | 0.45 | 2.67(2.46) |
| 1027 | 1027 | 1027 | 922.49 | 31.93 | 0.26 | 1.69(1.72) | 0.58 | 2.11(2.06) |
| 1037 | | | 922.56 | 22.89 | 0.27 | 2.15(2.07) | 0.55 | 2.25(2.26) |
| 1535 | | | 958.57 | 22.68 | 0.49 | 2.20(2.15) | 0.29 | 1.87(1.86) |
| 1564 | | | 960.53 | 24.07 | 0.19 | 1.83(1.88) | 0.39 | 2.38(2.55) |
| 1580 | | | 961.51 | 31.62 | 0.29 | 2.08(2.10) | 0.52 | 2.13(2.16) |
| 1586 | | | 962.5 | 30.81 | 0.25 | 2.02(2.09) | 0.48 | 2.34(2.38) |
| 1600 | | | 963.53 | 31.23 | 0.49 | 2.27(2.44) | 0.26 | 2.10(2.16) |
| 1804 | | | 977.47 | 32.28 | 0.12 | 2.14(1.96) | 0.29 | 2.25(1.98) |
| 1826 | 1826 | | 978.58 | 30.91 | 0.68 | 2.67(2.76) | 0.45 | 2.27(2.16) |
| 1843 | | | 980.46 | 40.55 | 0.36 | 2.20(2.31) | 0.16 | 1.34(1.14) |
| 2006 | | | 992.52 | 20.98 | 0.3 | 2.51(2.44) | 0.48 | 2.91(3.07) |
| 2039 | | | 994.56 | 31.26 | 0.41 | 1.87(1.84) | 0.16 | 2.03(1.93) |
| 2161 | | | 1003.55 | 31.38 | 0.42 | 2.34(2.42) | 0.19 | 1.77(1.61) |
| 2209 | | | 1006.56 | 31.67 | 0.23 | 1.58(1.38) | 0.06 | 2.17(2.17) |
| 2215 | | | 1006.61 | 22.98 | 0.26 | 2.37(2.12) | 0.1 | 1.72(1.72) |
| 2378 | | | 1018.55 | 31.65 | 0.25 | 2.17(2.36) | 0.42 | 2.61(2.64) |
| 2809 | | | 1049.59 | 31.98 | 0.45 | 2.37(2.51) | 0.26 | 2.12(1.80) |
| 2861 | 2861 | 2861 | 1053.58 | 32.42 | 0.6 | 2.41(2.44) | 0.29 | 2.07(2.09) |
| 2993 | | | 1063.61 | 26.2 | 0.23 | 1.85(1.89) | 0.06 | 1.80(1.80) |
| 3252 | | | 1080.6 | 33.13 | 0.62 | 3.19(3.34) | 0.42 | 3.09(3.43) |
| 3461 | 3461 | | 1094.67 | 32.95 | 0.48 | 3.07(3.16) | 0.28 | 2.34(2.19) |
| 3605 | 3605 | 3605 | 1104.63 | 25.66 | 0.21 | 2.23(2.28) | 0.52 | 2.39(2.46) |
| 3723 | | | 1113.63 | 24.53 | 0.41 | 2.66(2.74) | 0.58 | 3.35(3.24) |
| 3890 | 3890 | | 1127.58 | 26.87 | 0.08 | 1.53(1.61) | 0.32 | 1.94(2.02) |
| 4006 | | | 1136.68 | 26.87 | 0.26 | 1.95(1.80) | 0.06 | 0.90(0.90) |
| 4079 | | | 1143.64 | 33 | 0.34 | 2.42(2.48) | 0.16 | 1.96(1.95) |
| 4162 | | | 1150.63 | 33.28 | 0.4 | 2.63(2.62) | 0.19 | 1.98(2.15) |
| 4224 | | | 1156.64 | 33.16 | 0.52 | 2.53(2.72) | 0.26 | 2.51(2.57) |
| 4809 | 4809 | | 1209.63 | 33.63 | 0.21 | 2.11(1.87) | 0.48 | 2.12(2.35) |
| 4815 | | | 1210.66 | 33.55 | 0.18 | 1.72(1.76) | 0.39 | 2.07(1.97) |
| 4847 | 4847 | 4847 | 1213.62 | 24.59 | 0.31 | 3.09(3.25) | 0.5 | 3.18(2.90) |
| 4939 | | | 1225.67 | 25.29 | 0.26 | 3.45(3.49) | 0.48 | 3.92(3.97) |
| 4979 | | | 1229.69 | 34.21 | 0.58 | 2.50(2.52) | 0.39 | 2.07(2.18) |
| 5032 | | | 1235.66 | 33.71 | 0.26 | 2.41(2.32) | 0.45 | 2.43(2.43) |
| 5145 | | | 1247.71 | 34.45 | 0.37 | 2.21(2.15) | 0.16 | 2.18(2.23) |
| 5182 | | | 1251.71 | 25.66 | 0.33 | 2.66(2.70) | 0.55 | 3.38(3.42) |
| 5448 | 5448 | 5448 | 1278.79 | 35 | 0.19 | 2.62(2.64) | 0.55 | 3.49(3.57) |
| 5519 | | | 1285.71 | 33.91 | 0.47 | 2.54(2.63) | 0.61 | 2.94(2.97) |
| 5864 | | | 1324.69 | 33.96 | 0.37 | 2.91(2.99) | 0.16 | 2.63(2.75) |
| 5902 | | | 1328.58 | 28.1 | 0.12 | 3.20(3.29) | 0.32 | 3.06(2.85) |
| 5936 | 5936 | 5936 | 1332.7 | 34.13 | 0.3 | 2.60(2.49) | 0.68 | 2.51(2.47) |
| 5998 | 5998 | | 1339.69 | 34.02 | 0.25 | 2.16(1.91) | 0.55 | 2.30(2.28) |
| 6004 | | | 1339.75 | 26.79 | 0.56 | 2.85(2.97) | 0.35 | 2.61(2.72) |
| 6012 | | | 1340.7 | 34.54 | 0.36 | 2.47(2.54) | 0.13 | 2.54(2.50) |
| 6289 | 6289 | 6289 | 1372.72 | 34.27 | 0.44 | 3.53(3.54) | 0.71 | 3.35(3.48) |
| 6330 | | | 1377.81 | 34.85 | 0.33 | 2.48(2.57) | 0.45 | 3.50(3.66) |
| 6333 | 6333 | 6333 | 1377.86 | 35.22 | 0.18 | 2.86(3.00) | 0.47 | 3.43(3.65) |
| 6387 | | | 1382.72 | 34.44 | 0.33 | 2.23(2.30) | 0.13 | 2.36(2.52) |
| 6796 | | | 1430.81 | 27.22 | 0.68 | 3.13(3.39) | 0.77 | 3.69(3.76) |
| 7010 | 7010 | | 1456.67 | 23.22 | 0.14 | 2.80(2.69) | 0.39 | 2.71(2.90) |
| 7023 | 7023 | | 1457.76 | 34.7 | 0.27 | 2.26(2.13) | 0.52 | 2.46(2.31) |
| 7101 | 7101 | 7101 | 1466.75 | 34.88 | 0.44 | 3.01(3.09) | 0.28 | 2.71(2.75) |
| 7143 | 7143 | 7143 | 1470.82 | 28.49 | 0.49 | 3.00(3.12) | 0.29 | 2.15(2.28) |
| 7183 | 7183 | 7183 | 1474.83 | 28.06 | 0.17 | 2.25(2.22) | 0.31 | 2.51(2.38) |
| 7214 | | | 1477.76 | 26.9 | 0.59 | 3.27(3.38) | 0.71 | 3.86(4.01) |
| 7217 | 7217 | | 1477.75 | 34.81 | 0.3 | 2.36(2.39) | 0.52 | 2.89(2.90) |
| 7238 | 7238 | 7238 | 1479.85 | 34.98 | 0.53 | 2.94(2.97) | 0.28 | 2.81(2.86) |
| 7266 | | | 1482.88 | 35.1 | 0.44 | 3.33(3.17) | 0.58 | 4.57(4.62) |
| 7404 | | | 1499.79 | 35.06 | 0.62 | 2.85(2.80) | 0.45 | 2.47(2.52) |
| 7476 | 7476 | | 1509.7 | 26.24 | 0.14 | 2.62(2.53) | 0.39 | 2.50(2.74) |
| 7613 | | | 1525.74 | 34.48 | 0.29 | 2.27(2.13) | 0.45 | 3.13(3.04) |
| 7636 | 7636 | 7636 | 1528.81 | 27.47 | 0.67 | 3.79(3.93) | 0.94 | 4.05(4.30) |
| 7874 | | | 1557.87 | 28.16 | 0.49 | 3.44(3.68) | 0.29 | 2.66(2.65) |
| 7907 | | | 1562.83 | 35.24 | 0.15 | 2.05(2.26) | 0.35 | 1.84(1.66) |
| 8023 | 8023 | 8023 | 1575.87 | 27.62 | 0.1 | 2.27(2.09) | 0.42 | 2.53(2.32) |
| 8057 | | | 1579.83 | 35.27 | 0.23 | 2.16(2.09) | 0.42 | 2.65(2.84) |
| 8094 | | | 1584.9 | 28.16 | 0.23 | 2.80(2.91) | 0.06 | 1.95(1.95) |
| 8112 | | | 1587.82 | 28.34 | 0.45 | 2.47(2.45) | 0.26 | 2.11(2.05) |
| 8176 | | | 1595.82 | 35.22 | 0.23 | 2.23(2.17) | 0.45 | 2.02(1.78) |
| 8182 | 8182 | 8182 | 1596.84 | 35.53 | 0.56 | 2.65(2.54) | 0.29 | 2.78(2.83) |
| 8339 | | | 1614.91 | 28.79 | 0.71 | 2.97(2.93) | 0.48 | 2.87(2.85) |
| 8491 | 8491 | 8491 | 1632.83 | 35.36 | 0.27 | 2.87(3.04) | 0.52 | 3.23(3.35) |
| 8619 | | | 1646.88 | 28.02 | 0.14 | 2.80(2.97) | 0.32 | 2.68(2.56) |
| 8635 | | | 1648.85 | 35.47 | 0.38 | 2.56(2.70) | 0.16 | 3.05(3.30) |
| 8880 | 8880 | | 1678.92 | 35.48 | 0.11 | 2.72(2.78) | 0.32 | 3.44(3.58) |
| 8919 | | | 1683.89 | 35.66 | 0.27 | 2.22(2.16) | 0.48 | 2.62(2.44) |
| 9085 | 9085 | | 1704.89 | 34.11 | 0.29 | 2.31(2.36) | 0.1 | 2.25(2.23) |
| 9106 | | | 1707.01 | 23.73 | 0.21 | 2.45(2.42) | 0.03 | 2.08(2.08) |
| 9380 | | | 1742.9 | 35.61 | 0.38 | 2.59(2.75) | 0.65 | 2.58(2.58) |
| 9648 | | | 1774.97 | 30.5 | 0.44 | 2.49(2.49) | 0.19 | 2.39(2.38) |
| 9802 | | | 1794.92 | 35.89 | 0.15 | 1.58(1.49) | 0.32 | 2.05(2.20) |
| 9956 | | | 1813.98 | 36.09 | 0.51 | 2.95(2.92) | 0.29 | 2.51(2.49) |
| 10067 | | | 1829.94 | 36.26 | 0.27 | 2.26(2.22) | 0.1 | 1.17(1.13) |
| 10296 | | | 1857.98 | 36.06 | 0.11 | 2.06(2.12) | 0.26 | 2.63(2.49) |
| 10349 | | | 1865.97 | 36.26 | 0.1 | 2.55(2.83) | 0.29 | 1.75(1.91) |
| 10913 | | | 1946.07 | 30.85 | 0.45 | 2.51(2.50) | 0.26 | 2.20(2.30) |
| 10992 | 10992 | | 1958.05 | 36.7 | 0.11 | 2.12(2.19) | 0.35 | 2.33(2.51) |
| 11096 | | | 1972.08 | 25.48 | 0.27 | 2.43(2.27) | 0.1 | 2.00(2.06) |
| 11577 | | | 2044.12 | 32.14 | 0.11 | 2.04(2.42) | 0.32 | 2.19(2.05) |
| 12188 | | | 2139.02 | 25.31 | 0.33 | 2.63(2.76) | 0.48 | 3.30(3.20) |
| 12274 | 12274 | 12274 | 2150.19 | 25.91 | 0.27 | 2.13(1.98) | 0.55 | 2.58(2.54) |
| 12534 | 12534 | | 2184.13 | 37.02 | 0.26 | 1.96(1.92) | 0.03 | 0.85(0.85) |
| 12814 | | | 2224.15 | 37.58 | 0.16 | 2.20(2.27) | 0.35 | 2.30(2.47) |
| 12829 | | | 2226.23 | 32.28 | 0.37 | 2.87(2.96) | 0.19 | 2.50(2.45) |
| 13109 | 13109 | | 2267.24 | 32.44 | 0.22 | 2.28(2.39) | 0 | 0.00(0.00) |
| 13345 | | | 2300.1 | 31.84 | 0.25 | 2.32(2.52) | 0.42 | 3.03(3.24) |
| 13670 | 13670 | 13670 | 2346.31 | 33.26 | 0.25 | 2.48(2.41) | 0.45 | 2.46(2.61) |
| 14143 | | | 2423.28 | 36.64 | 0.23 | 2.13(1.95) | 0.42 | 3.12(2.96) |
| 14373 | | | 2459.3 | 33.69 | 0.21 | 1.93(2.13) | 0.42 | 2.28(2.54) |
| 14469 | 14469 | | 2476.33 | 28.89 | 0.11 | 1.58(1.65) | 0.32 | 1.96(1.73) |
| 14517 | | | 2483.25 | 38.39 | 0.19 | 1.73(1.77) | 0.35 | 2.16(2.30) |
| 14710 | 14710 | | 2512.25 | 32.75 | 0.1 | 2.53(2.29) | 0.39 | 2.86(2.90) |
| 14835 | 14835 | 14835 | 2530.37 | 33.59 | 0.18 | 2.12(2.17) | 0.58 | 2.72(2.75) |
| 16361 | | | 2796.53 | 24.95 | 0.32 | 3.24(3.32) | 0.13 | 2.40(2.54) |
| 16428 | 16428 | 16428 | 2809.53 | 20.89 | 0.36 | 2.68(2.87) | 0.1 | 2.43(2.65) |
| 16759 | | | 2867.52 | 31.06 | 0.27 | 2.66(2.88) | 0.1 | 1.06(1.16) |
| 17267 | | | 2966.56 | 29.03 | 0.33 | 3.26(3.51) | 0.13 | 3.70(3.74) |
| 18815 | 18815 | 18815 | 3307.78 | 23.81 | 0.29 | 2.87(2.69) | 0.06 | 3.33(3.33) |
| 18891 | | | 3325.81 | 23.82 | 0.41 | 3.65(3.74) | 0.19 | 3.23(3.39) |
| 21184 | 21184 | | 4101.2 | 26.01 | 0.16 | 2.54(2.50) | 0.39 | 2.55(2.56) |
| 21239 | | | 4119.18 | 25.8 | 0.45 | 3.93(4.09) | 0.23 | 4.08(3.95) |
| 22284 | | | 4563.5 | 26.41 | 0.36 | 3.56(3.58) | 0.16 | 2.76(2.84) |
| 149512 | | | 1485.85 | 22.56 | 0.1 | 2.57(2.47) | 0.26 | 2.44(2.48) |
| 149766 | 149766 | 149766 | 846.43 | 21.51 | 0.1 | 1.28(1.24) | 0.42 | 2.34(2.20) |
| 149777 | | | 852.48 | 24.22 | 0.19 | 1.12(1.08) | 0.35 | 1.89(1.80) |
| 149780 | | | 855.47 | 23.41 | 0.15 | 2.11(2.12) | 0.39 | 2.36(2.48) |
| 149790 | 149790 | 149790 | 862.4 | 21.52 | 0.07 | 1.82(2.31) | 0.35 | 2.53(2.28) |
| 149814 | | | 880.45 | 24.08 | 0.05 | 1.12(1.07) | 0.23 | 1.58(1.66) |
| 149852 | 149852 | 149852 | 899.43 | 29.06 | 0.01 | 2.10(2.10) | 0.26 | 2.06(1.93) |
| 149882 | | | 919.51 | 32.26 | 0.05 | 1.42(1.46) | 0.23 | 2.37(2.26) |
| 149899 | | | 929.44 | 25.88 | 0.15 | 1.62(1.56) | 0.32 | 2.14(1.97) |
| 149911 | | | 938.54 | 30.53 | 0.14 | 1.46(1.36) | 0.29 | 2.43(2.46) |
| 149938 | 149938 | | 959.54 | 31.51 | 0.23 | 2.30(2.32) | 0 | 0.00(0.00) |
| 149948 | | | 969.5 | 26.06 | 0.1 | 2.37(2.33) | 0.29 | 2.30(2.20) |
| 149985 | | | 994.55 | 23.27 | 0.08 | 1.88(1.59) | 0.26 | 2.00(2.03) |
| 149987 | | | 996.48 | 41.3 | 0.25 | 1.99(1.91) | 0.06 | 1.52(1.52) |
| 149992 | 149992 | | 1001.55 | 32.15 | 0.26 | 2.13(2.28) | 0 | 0.00(0.00) |
| 150014 | | | 1016.65 | 20.47 | 0.23 | 1.69(1.67) | 0.03 | 0.63(0.63) |
| 150040 | 150040 | | 1029.59 | 32.62 | 0.42 | 2.02(2.18) | 0.1 | 2.68(2.42) |
| 150041 | | | 1032.6 | 23.29 | 0.59 | 2.64(2.66) | 0.32 | 2.55(2.44) |
| 150043 | 150043 | | 1032.67 | 34.77 | 0.04 | 1.70(1.54) | 0.26 | 2.29(2.58) |
| 150052 | | | 1038.58 | 25.76 | 0.1 | 2.12(2.09) | 0.26 | 1.77(1.60) |
| 150056 | | | 1042.51 | 41.52 | 0.23 | 2.07(2.15) | 0.03 | 1.12(1.12) |
| 150110 | | | 1086.59 | 32.51 | 0.25 | 2.12(2.02) | 0.06 | 2.03(2.03) |
| 150125 | | | 1099.6 | 32.49 | 0.38 | 2.27(2.29) | 0.16 | 2.58(2.74) |
| 150126 | | | 1099.67 | 24.2 | 0.23 | 2.41(2.37) | 0.39 | 3.27(3.24) |
| 150136 | | | 1109.6 | 32.95 | 0.53 | 2.24(2.29) | 0.26 | 2.31(2.39) |
| 150142 | | | 1114.64 | 24 | 0.49 | 2.70(2.82) | 0.26 | 2.32(2.45) |
| 150144 | | | 1116.68 | 32.85 | 0.1 | 2.38(2.24) | 0.26 | 1.93(2.10) |
| 150167 | | | 1134.6 | 32.81 | 0.29 | 2.49(2.46) | 0.06 | 1.57(1.57) |
| 150185 | 150185 | 150185 | 1149.75 | 35.14 | 0.15 | 2.67(2.55) | 0.45 | 3.12(3.28) |
| 150189 | 150189 | 150189 | 1155.56 | 25.94 | 0.05 | 2.06(1.98) | 0.32 | 2.60(2.55) |
| 150194 | | | 1158.63 | 33.34 | 0.18 | 2.20(2.39) | 0.42 | 2.22(2.41) |
| 150208 | | | 1176.61 | 33.47 | 0.08 | 1.98(1.99) | 0.23 | 2.50(2.33) |
| 150210 | | | 1176.7 | 21.69 | 0.26 | 1.84(1.79) | 0.06 | 1.58(1.58) |
| 150222 | | | 1184.71 | 20.71 | 0.23 | 2.00(1.97) | 0.06 | 1.47(1.47) |
| 150225 | | | 1190.68 | 24.33 | 0.26 | 2.09(2.23) | 0.03 | 2.63(2.63) |
| 150231 | | | 1198.68 | 25.3 | 0.14 | 2.50(2.46) | 0.32 | 2.50(2.55) |
| 150252 | | | 1229.49 | 28.88 | 0.11 | 3.09(3.17) | 0.32 | 3.57(3.53) |
| 150280 | | | 1268.67 | 33.52 | 0.12 | 2.43(2.56) | 0.29 | 2.25(2.44) |
| 150294 | | | 1276.67 | 28.22 | 0.25 | 1.93(1.89) | 0.06 | 1.97(1.97) |
| 150304 | 150304 | | 1287.62 | 25.99 | 0.05 | 3.45(3.52) | 0.26 | 3.64(3.71) |
| 150307 | 150307 | | 1291.72 | 25.97 | 0.19 | 2.42(2.56) | 0.45 | 2.81(3.06) |
| 150323 | | | 1305.7 | 25.99 | 0.08 | 2.12(2.49) | 0.23 | 2.50(2.44) |
| 150334 | | | 1318.84 | 19.65 | 0.23 | 1.75(1.89) | 0.03 | 2.24(2.24) |
| 150338 | 150338 | | 1322.6 | 34.3 | 0.03 | 2.81(2.81) | 0.23 | 3.04(2.92) |
| 150355 | 150355 | 150355 | 1338.69 | 33.88 | 0.07 | 1.91(1.50) | 0.32 | 2.50(2.29) |
| 150364 | | | 1349.51 | 29.72 | 0.07 | 2.83(2.78) | 0.23 | 3.09(2.78) |
| 150369 | | | 1355.78 | 27.96 | 0.3 | 2.44(2.52) | 0.1 | 1.63(1.45) |
| 150370 | 150370 | | 1357.59 | 23.86 | 0.04 | 1.94(1.63) | 0.23 | 2.81(2.82) |
| 150374 | | | 1362.69 | 34.33 | 0.18 | 2.38(2.16) | 0.35 | 2.70(2.90) |
| 150396 | | | 1385.77 | 26.75 | 0.07 | 1.85(2.21) | 0.23 | 2.41(2.78) |
| 150397 | 150397 | | 1386.67 | 23.7 | 0.19 | 2.56(2.34) | 0.45 | 2.95(3.12) |
| 150403 | 150403 | 150403 | 1395.85 | 34.56 | 0.07 | 3.25(3.59) | 0.35 | 4.24(4.26) |
| 150411 | | | 1400.77 | 26.65 | 0.07 | 2.55(2.60) | 0.23 | 2.37(2.35) |
| 150416 | | | 1406.71 | 34.31 | 0.19 | 2.58(2.56) | 0.42 | 2.24(2.44) |
| 150418 | | | 1408.79 | 26.71 | 0.07 | 2.65(3.19) | 0.23 | 2.01(1.85) |
| 150423 | | | 1413.66 | 25.58 | 0.08 | 2.76(2.69) | 0.26 | 3.38(3.55) |
| 150428 | | | 1415.76 | 34.6 | 0.42 | 2.72(2.99) | 0.19 | 2.74(2.70) |
| 150430 | | | 1416.78 | 27.16 | 0.08 | 1.88(1.84) | 0.26 | 2.39(2.42) |
| 150436 | 150436 | | 1420.75 | 34.67 | 0.07 | 2.71(2.26) | 0.29 | 2.77(2.56) |
| 150440 | | | 1424.68 | 22.13 | 0.05 | 1.91(2.00) | 0.23 | 2.56(2.47) |
| 150480 | | | 1460.81 | 27.06 | 0.14 | 2.47(2.47) | 0.35 | 2.34(2.30) |
| 150491 | | | 1469.79 | 34.98 | 0.36 | 2.01(1.98) | 0.13 | 2.43(2.52) |
| 150508 | | | 1482.82 | 22.3 | 0.1 | 3.02(2.67) | 0.29 | 3.54(3.40) |
| 150513 | | | 1486.83 | 27.62 | 0.4 | 2.86(2.99) | 0.23 | 2.39(2.38) |
| 150541 | | | 1514.71 | 23.04 | 0.1 | 2.90(2.94) | 0.29 | 3.88(3.69) |
| 150545 | | | 1519.82 | 35.32 | 0.21 | 2.70(2.90) | 0 | 0.00(0.00) |
| 150585 | | | 1562.88 | 27.73 | 0.23 | 3.03(3.01) | 0.06 | 2.86(2.86) |
| 150634 | | | 1606.83 | 35.56 | 0.05 | 2.13(2.13) | 0.23 | 2.32(2.19) |
| 150648 | | | 1616.84 | 35.29 | 0.16 | 1.96(2.07) | 0.32 | 2.80(2.54) |
| 150662 | | | 1626.83 | 35.38 | 0.1 | 1.83(2.20) | 0.26 | 2.17(2.17) |
| 150672 | | | 1642.87 | 28.24 | 0.08 | 1.96(1.87) | 0.23 | 2.32(2.37) |
| 150688 | | | 1666.82 | 23.69 | 0.19 | 2.35(2.40) | 0.42 | 2.85(2.87) |
| 150697 | 150697 | 150697 | 1674.88 | 35.85 | 0.08 | 1.96(1.70) | 0.32 | 3.36(3.22) |
| 150703 | | | 1677.9 | 35.62 | 0.23 | 2.39(2.43) | 0.03 | 2.90(2.90) |
| 150717 | | | 1692.89 | 35.63 | 0.11 | 1.96(1.55) | 0.26 | 2.22(2.30) |
| 150754 | | | 1723.83 | 22.06 | 0.07 | 2.15(2.14) | 0.23 | 2.26(2.71) |
| 150776 | | | 1741.95 | 28.77 | 0.14 | 2.95(2.80) | 0.32 | 2.62(2.54) |
| 150795 | | | 1761.95 | 30.02 | 0.38 | 2.39(2.41) | 0.13 | 2.46(1.96) |
| 150806 | 150806 | 150806 | 1771.88 | 24.48 | 0.1 | 2.37(2.27) | 0.39 | 2.69(2.71) |
| 150841 | 150841 | | 1806.03 | 29.26 | 0.1 | 2.29(2.31) | 0.32 | 2.48(2.27) |
| 150844 | | | 1807 | 29 | 0.18 | 2.35(2.18) | 0.42 | 2.55(2.53) |
| 150884 | | | 1842.03 | 24.48 | 0.25 | 2.35(2.25) | 0.06 | 1.80(1.80) |
| 150938 | 150938 | | 1894.03 | 30.33 | 0.1 | 2.47(2.59) | 0.35 | 2.41(2.49) |
| 150943 | 150943 | | 1899.93 | 21.61 | 0.07 | 2.99(3.07) | 0.29 | 3.60(3.94) |
| 150947 | | | 1904.02 | 30.7 | 0.07 | 1.44(1.59) | 0.23 | 2.61(2.16) |
| 150977 | | | 1928.99 | 24.65 | 0.15 | 2.28(2.08) | 0.35 | 2.45(2.52) |
| 151035 | | | 1989.99 | 25.53 | 0.04 | 2.80(2.93) | 0.23 | 1.75(1.72) |
| 151037 | 151037 | | 1991.05 | 30.56 | 0.1 | 1.64(1.53) | 0.32 | 2.41(2.34) |
| 151064 | | | 2025.06 | 37.05 | 0.11 | 1.96(2.06) | 0.26 | 2.26(2.06) |
| 151068 | | | 2028.1 | 31.44 | 0.12 | 2.20(1.75) | 0.29 | 2.06(1.98) |
| 151089 | | | 2049 | 21.31 | 0.15 | 2.82(3.16) | 0.32 | 3.07(2.98) |
| 151103 | 151103 | | 2063.89 | 26.69 | 0.03 | 1.69(1.69) | 0.23 | 2.55(2.57) |
| 151133 | | | 2099.13 | 31.18 | 0.14 | 2.02(1.78) | 0.35 | 2.49(2.59) |
| 151136 | 151136 | 151136 | 2102.92 | 21.77 | 0.03 | 2.00(2.00) | 0.26 | 3.21(3.09) |
| 151174 | 151174 | | 2138.24 | 22.75 | 0.25 | 2.51(2.37) | 0 | 0.00(0.00) |
| 151191 | | | 2156.17 | 26.24 | 0.1 | 2.36(2.38) | 0.29 | 2.33(2.34) |
| 151213 | 151213 | | 2185.21 | 25.49 | 0.03 | 1.25(1.25) | 0.23 | 2.32(2.23) |
| 151220 | | | 2189.95 | 22.11 | 0.05 | 3.02(2.80) | 0.23 | 2.93(3.11) |
| 151238 | | | 2206.14 | 32.39 | 0.16 | 2.46(2.48) | 0.32 | 2.95(3.26) |
| 151239 | | | 2207.18 | 31.87 | 0.07 | 1.97(1.97) | 0.23 | 2.22(2.14) |
| 151245 | 151245 | | 2218.19 | 25.46 | 0.05 | 1.61(1.67) | 0.26 | 2.63(2.58) |
| 151276 | | | 2255.26 | 23.33 | 0.07 | 1.43(1.24) | 0.23 | 2.83(2.58) |
| 151277 | 151277 | | 2258.2 | 38.01 | 0.11 | 1.46(1.53) | 0.32 | 2.73(2.73) |
| 151278 | | | 2260.03 | 20.82 | 0.05 | 2.21(2.39) | 0.23 | 3.29(3.75) |
| 151295 | 151295 | | 2289.01 | 23.09 | 0.05 | 2.60(2.76) | 0.29 | 2.73(3.00) |
| 151296 | | | 2293.16 | 37.42 | 0.1 | 1.43(1.45) | 0.26 | 1.68(1.87) |
| 151319 | | | 2320.25 | 26.49 | 0.41 | 2.42(2.41) | 0.16 | 2.44(2.53) |
| 151336 | | | 2344.16 | 22.96 | 0.1 | 2.75(2.74) | 0.26 | 3.25(3.35) |
| 151342 | 151342 | | 2353.08 | 26.82 | 0.03 | 2.73(2.73) | 0.23 | 1.80(1.91) |
| 151350 | | | 2367.26 | 26.97 | 0.1 | 2.01(1.94) | 0.26 | 2.33(2.30) |
| 151356 | | | 2374.27 | 33.06 | 0.26 | 2.17(2.42) | 0.06 | 1.66(1.66) |
| 151358 | 151358 | | 2376.04 | 23.34 | 0.08 | 3.08(3.14) | 0.29 | 3.48(3.74) |
| 151364 | | | 2384.27 | 37.98 | 0.11 | 1.66(1.74) | 0.26 | 2.42(2.40) |
| 151366 | | | 2385.31 | 33.5 | 0.11 | 1.89(1.89) | 0.32 | 2.31(1.97) |
| 151373 | | | 2397.24 | 27.05 | 0.08 | 1.85(1.69) | 0.26 | 2.62(2.53) |
| 151378 | | | 2401.24 | 32.19 | 0.07 | 1.57(1.24) | 0.23 | 2.17(1.94) |
| 151397 | | | 2434.31 | 33.21 | 0.14 | 1.99(2.08) | 0.29 | 2.59(2.41) |
| 151406 | | | 2459.17 | 21.27 | 0.07 | 2.49(2.93) | 0.23 | 3.16(3.03) |
| 151413 | | | 2474.34 | 24.18 | 0.07 | 2.40(2.58) | 0.26 | 2.16(2.27) |
| 151424 | | | 2489.11 | 23.54 | 0.11 | 2.52(2.66) | 0.29 | 2.84(3.22) |
| 151425 | | | 2491.28 | 33.75 | 0.21 | 2.42(2.39) | 0 | 0.00(0.00) |
| 151444 | 151444 | | 2521.21 | 22.84 | 0.04 | 2.26(2.34) | 0.23 | 2.59(2.58) |
| 151446 | | | 2524.34 | 27.1 | 0.18 | 1.84(1.93) | 0.35 | 2.28(2.26) |
| 151492 | | | 2617.25 | 21.28 | 0.07 | 3.25(2.97) | 0.23 | 3.37(3.41) |
| 151495 | 151495 | | 2620.35 | 25.03 | 0.03 | 1.55(1.55) | 0.23 | 2.14(2.07) |
| 151499 | | | 2633.32 | 23.53 | 0.1 | 2.55(3.10) | 0.29 | 3.17(2.90) |
| 151510 | | | 2658.45 | 24.48 | 0.11 | 1.77(1.59) | 0.26 | 2.02(2.16) |
| 151527 | | | 2697.34 | 31.9 | 0.26 | 2.05(2.25) | 0.1 | 0.87(0.64) |
| 151589 | | | 2927.44 | 35.19 | 0.07 | 2.20(2.29) | 0.26 | 2.48(2.97) |
| 151598 | | | 2987.61 | 30.05 | 0.08 | 1.77(1.73) | 0.23 | 2.08(2.42) |
| 151624 | 151624 | 151624 | 3076.51 | 29.21 | 0.01 | 2.07(2.07) | 0.26 | 2.66(2.33) |
| 151625 | 151625 | 151625 | 3077.76 | 25.68 | 0.07 | 2.05(2.24) | 0.32 | 2.41(2.33) |
| 151639 | 151639 | 151639 | 3165.64 | 32.95 | 0.11 | 1.94(2.06) | 0.39 | 3.29(3.40) |
| 151644 | | | 3209.69 | 25.15 | 0.07 | 2.92(3.08) | 0.26 | 2.71(2.82) |
| 151659 | 151659 | | 3306.75 | 27.03 | 0.07 | 2.55(2.10) | 0.26 | 3.28(3.26) |
| 151664 | 151664 | 151664 | 3364.74 | 35.47 | 0.05 | 1.82(1.77) | 0.32 | 2.38(2.44) |
| 151667 | | | 3376.83 | 34.68 | 0.16 | 2.57(2.71) | 0.39 | 3.12(3.07) |
| 151669 | 151669 | 151669 | 3392.79 | 34.29 | 0.03 | 1.43(1.43) | 0.32 | 2.57(2.84) |
| 151675 | 151675 | | 3436.69 | 30.69 | 0.16 | 2.62(2.54) | 0.42 | 2.99(2.80) |
| 151680 | 151680 | | 3463.77 | 34.53 | 0.07 | 2.13(1.78) | 0.29 | 2.27(2.27) |
| 151682 | | | 3472.87 | 24.13 | 0.4 | 3.05(3.07) | 0.19 | 2.46(2.55) |
| 151699 | | | 3641.93 | 31.8 | 0.12 | 2.85(2.81) | 0.32 | 2.43(2.38) |
| 151701 | | | 3704.94 | 24.77 | 0.1 | 2.78(2.56) | 0.26 | 3.37(3.33) |
| 151703 | | | 3800.95 | 20.9 | 0.07 | 3.29(3.46) | 0.23 | 3.11(3.15) |
| 151705 | 151705 | | 3914.05 | 20.9 | 0.04 | 2.36(2.35) | 0.26 | 2.93(2.95) |
| 151713 | 151713 | 151713 | 4074.92 | 24.22 | 0.04 | 3.61(3.68) | 0.32 | 2.57(2.46) |
| 151714 | | | 4117.99 | 20.87 | 0.07 | 3.53(3.42) | 0.23 | 3.39(3.58) |
| 151717 | 151717 | | 4134.22 | 26.28 | 0.08 | 2.73(2.75) | 0.32 | 2.95(2.62) |
| 151719 | | | 4137.12 | 25.07 | 0.08 | 3.44(3.47) | 0.23 | 3.37(3.29) |
| 151725 | | | 4265.19 | 23.12 | 0.07 | 3.62(3.43) | 0.26 | 3.08(3.00) |
| 151731 | | | 4323.23 | 23.2 | 0.07 | 2.17(1.88) | 0.23 | 2.05(2.01) |
| 151735 | | | 4478.96 | 21.22 | 0.08 | 2.87(2.83) | 0.23 | 3.66(3.61) |
| 151742 | 151742 | | 4716.31 | 23.28 | 0.03 | 3.07(3.07) | 0.23 | 2.71(2.61) |
| 151745 | 151745 | 151745 | 4800.01 | 28.72 | 0.18 | 2.57(2.72) | 0.48 | 2.83(3.08) |
| 151747 | | | 4872.26 | 21.91 | 0.08 | 3.30(3.15) | 0.23 | 3.33(3.61) |
| 151751 | | | 5042.37 | 22.01 | 0.1 | 3.38(3.45) | 0.26 | 3.05(3.29) |
| 151752 | | | 5080.47 | 24.72 | 0.08 | 2.45(2.11) | 0.23 | 3.15(3.25) |
| 151757 | | | 5208.59 | 23.05 | 0.08 | 2.93(2.79) | 0.23 | 2.74(2.64) |

TABLE 2a
List of markers that enable the differentiation of a cholangiocellular
carcinoma from benign strictures, especially from a primary sclerosing cholangitis,
in body fluid samples, for example, in urine, in a multimarker model.

| | | | | | Cholangiocellular | | | |
|---|---|---|---|---|---|---|---|---|
| | | | | | carcinoma | | Benign stricture | |
| | | more | Mass | CE time | | mean log amp | | mean log amp |
| Protein ID | preferably | preferably | [Da] | [min] | Frequency | (log median) | Frequency | (log median) |
| 6498 | | | 923.42 | 22.01 | 0.34 | 1.74(1.82) | 0.69 | 1.89(1.90) |
| 8503 | 8503 | 8503 | 947.47 | 24.74 | 0.16 | 2.06(2.13) | 0.37 | 2.04(2.06) |
| 11413 | | | 981.59 | 24.80 | 0.36 | 2.01(2.02) | 0.70 | 2.25(2.27) |
| 13746 | 13746 | 13746 | 1025.47 | 25 | 0.33 | 1.96(1.98) | 0.17 | 1.78(1.80) |
| 15776 | | | 1068.45 | 24.76 | 0.87 | 2.87(2.81) | 0.97 | 3.19(3.22) |
| 15800 | | | 1068.51 | 21.75 | 0.7 | 2.26(2.26) | 0.85 | 2.45(2.49) |
| 16854 | 16854 | | 1082.5 | 23.91 | 0.37 | 1.98(1.93) | 0.71 | 2.26(2.27) |
| 18939 | 18939 | | 1114.48 | 24.21 | 0.21 | 1.89(1.98) | 0.59 | 2.03(2.05) |
| 19773 | 19773 | 19773 | 1128.39 | 33.59 | 0.4 | 2.83(2.82) | 0.67 | 2.99(3.05) |
| 20334 | 20334 | 20334 | 1138.47 | 37.07 | 0.43 | 1.95(2.05) | 0.42 | 1.93(2.02) |
| 21709 | | | 1156.61 | 27.15 | 0.43 | 2.31(2.37) | 0.05 | 2.33(2.13) |
| 23628 | 23628 | 23628 | 1184.56 | 26.4 | 0.44 | 2.12(2.04) | 0.3 | 2.03(2.00) |
| 24393 | 24393 | 24393 | 1198.54 | 25.95 | 0.52 | 1.97(1.99) | 0.23 | 1.74(1.74) |
| 25866 | 25866 | | 1223.55 | 27.46 | 0.6 | 2.29(2.25) | 0.37 | 2.02(2.03) |
| 26431 | 26431 | 26431 | 1231.49 | 39.57 | 0.62 | 2.30(2.29) | 0.51 | 2.02(2.10) |
| 28103 | | | 1257.44 | 33.92 | 0.65 | 3.03(3.04) | 0.95 | 3.20(3.20) |
| 28306 | 28306 | 28306 | 1260.6 | 27.43 | 0.37 | 2.15(2.05) | 0.07 | 1.88(1.72) |
| 29906 | 29906 | | 1287.59 | 21.87 | 0.44 | 2.51(2.52) | 0.83 | 2.71(2.68) |
| 31480 | 31480 | | 1312.55 | 29.77 | 0.83 | 2.60(2.70) | 0.93 | 3.02(3.11) |
| 32470 | 32470 | | 1326.55 | 29.2 | 0.4 | 2.07(2.04) | 0.64 | 2.30(2.43) |
| 33727 | 33727 | 33727 | 1350.63 | 27.09 | 0.33 | 2.56(2.63) | 0.65 | 2.46(2.47) |
| 33840 | | | 1352.78 | 24.6 | 0.56 | 2.26(2.39) | 0.78 | 2.51(2.52) |
| 33973 | | | 1353.66 | 25.63 | 0.59 | 2.30(2.28) | 0.82 | 2.47(2.53) |
| 36156 | | | 1392.62 | 21.75 | 0.9 | 3.20(3.26) | 0.97 | 3.57(3.63) |
| 37056 | | | 1409.58 | 22.04 | 1 | 3.63(3.61) | 0.99 | 3.96(3.98) |
| 37949 | | | 1425.59 | 22.32 | 0.86 | 3.04(3.09) | 0.98 | 3.43(3.47) |
| 40091 | | | 1449.64 | 21.86 | 0.95 | 3.25(3.29) | 0.99 | 3.71(3.76) |
| 41514 | | | 1467.81 | 24.69 | 0.6 | 2.99(3.03) | 0.83 | 3.36(3.41) |
| 42304 | | | 1485.67 | 23.77 | 0.81 | 2.95(2.98) | 0.96 | 3.18(3.17) |
| 42404 | 42404 | 42404 | 1487.65 | 29.62 | 0.14 | 2.24(2.22) | 0.43 | 2.54(2.57) |
| 42833 | 42833 | | 1495.68 | 23.36 | 0.32 | 2.16(2.10) | 0.68 | 2.30(2.32) |
| 45980 | 45980 | | 1552.5 | 37.21 | 0.4 | 2.94(3.01) | 0.82 | 3.32(3.38) |
| 46338 | 46338 | | 1560.58 | 21.77 | 0.4 | 2.45(2.47) | 0.69 | 2.67(2.70) |
| 46649 | 46649 | 46649 | 1563.7 | 29.46 | 0.35 | 2.48(2.59) | 0.55 | 2.22(2.20) |
| 48093 | | | 1579.68 | 23 | 0.59 | 2.55(2.71) | 0.87 | 2.71(2.79) |
| 48580 | 48580 | | 1588.71 | 30.15 | 0.4 | 2.30(2.44) | 0.8 | 2.70(2.72) |
| 49958 | 49958 | 49958 | 1608.73 | 30.93 | 0.48 | 2.50(2.52) | 0.7 | 2.58(2.53) |
| 50212 | 50212 | | 1613.82 | 23.99 | 0.29 | 2.04(2.09) | 0.73 | 2.21(2.24) |
| 50638 | 50638 | 50638 | 1620.7 | 22.66 | 0.21 | 2.47(2.49) | 0.65 | 2.51(2.60) |
| 50904 | 50904 | 50904 | 1624.55 | 37.73 | 0.57 | 2.77(2.85) | 0.89 | 3.06(3.01) |
| 51804 | | | 1634.8 | 29.72 | 0.75 | 3.17(3.11) | 0.68 | 2.65(2.62) |
| 52189 | | | 1640.58 | 23.24 | 0.76 | 3.31(3.32) | 0.99 | 3.73(3.80) |
| 53216 | | | 1654.78 | 23.13 | 0.75 | 3.01(3.14) | 0.33 | 2.45(2.43) |
| 54687 | 54687 | 54687 | 1684.67 | 31.75 | 0.37 | 3.18(3.33) | 0.23 | 3.11(3.33) |
| 54846 | | | 1687.54 | 37.79 | 0.25 | 2.19(2.28) | 0.63 | 2.24(2.24) |
| 58880 | 58880 | | 1764.68 | 19.91 | 0.24 | 2.11(2.07) | 0.52 | 2.47(2.53) |
| 59928 | 59928 | 59928 | 1788.84 | 29.87 | 0.27 | 2.25(2.23) | 0.37 | 2.63(2.65) |
| 60259 | 60259 | 60259 | 1796.84 | 20.95 | 0.37 | 2.25(1.97) | 0.15 | 1.85(1.88) |
| 60816 | | | 1808.79 | 23.72 | 0.20 | 2.19(2.19) | 0.55 | 2.46(2.51) |
| 61221 | | | 1817.69 | 20.23 | 0.71 | 3.12(3.19) | 0.93 | 3.47(3.52) |
| 61332 | | | 1819.8 | 23.36 | 1 | 3.32(3.37) | 0.99 | 3.65(3.72) |
| 61984 | | | 1835.71 | 19.91 | 0.51 | 2.64(2.75) | 0.81 | 2.92(3.00) |
| 64899 | | | 1892.97 | 24.56 | 0.44 | 2.45(2.46) | 0.69 | 2.65(2.64) |
| 64905 | 64905 | | 1893.03 | 28.86 | 0.25 | 2.44(2.57) | 0.67 | 2.40(2.42) |
| 65746 | | | 1911.05 | 24.98 | 0.94 | 4.41(4.50) | 0.98 | 4.76(4.87) |
| 65998 | | | 1913.90 | 40.96 | 0.27 | 2.07(2.12) | 0.58 | 2.35(2.46) |
| 67632 | | | 1943.01 | 24.94 | 0.43 | 3.34(3.22) | 0.07 | 2.93(2.69) |
| 67951 | | | 1950.85 | 35.77 | 0.30 | 2.50(2.61) | 0.67 | 2.66(2.71) |
| 69769 | | | 1991.94 | 22.05 | 0.95 | 3.50(3.55) | 0.98 | 3.08(3.11) |
| 69979 | 69979 | | 1996.79 | 20.98 | 0.49 | 2.57(2.52) | 0.8 | 2.83(2.83) |
| 70413 | | | 2007.94 | 22.1 | 1 | 3.68(3.71) | 0.99 | 3.40(3.40) |
| 72161 | | | 2039.13 | 21.78 | 0.63 | 2.82(2.85) | 0.83 | 3.16(3.28) |
| 72317 | | | 2041.98 | 28.5 | 0.21 | 1.82(1.86) | 0.55 | 1.96(1.98) |
| 73434 | | | 2067.82 | 20.62 | 0.6 | 2.96(3.01) | 0.9 | 3.08(3.16) |
| 73697 | | | 2070.92 | 25.4 | 0.75 | 2.70(2.78) | 0.92 | 2.94(2.97) |
| 74420 | 74420 | 74420 | 2085.93 | 22.07 | 0.32 | 4.07(4.24) | 0.5 | 4.13(4.25) |
| 75025 | 75025 | | 2090.9 | 19.77 | 0.52 | 2.66(2.66) | 0.15 | 2.22(2.16) |
| 76839 | 76839 | 76839 | 2128.98 | 26.97 | 0.27 | 1.94(1.90) | 0.39 | 2.09(2.14) |
| 78111 | 78111 | 78111 | 2157.03 | 19.51 | 0.38 | 2.52(2.51) | 0.08 | 2.21(2.24) |
| 79720 | 79720 | 79720 | 2187.95 | 39.78 | 0.52 | 2.59(2.76) | 0.83 | 2.94(2.98) |
| 81263 | 81263 | 81263 | 2211.92 | 38.57 | 0.17 | 2.46(2.57) | 0.04 | 2.16(2.29) |
| 83107 | | | 2244.07 | 19.50 | 0.45 | 3.02(3.11) | 0.10 | 2.51(2.48) |
| 84302 | 84302 | | 2261.08 | 25.68 | 0.57 | 2.42(2.53) | 0.22 | 1.90(1.96) |
| 86426 | | | 2306.03 | 19.53 | 0.78 | 3.48(3.47) | 0.49 | 2.89(3.01) |
| 87411 | 87411 | | 2321.17 | 22.06 | 0.6 | 2.74(2.80) | 0.3 | 2.57(2.55) |
| 87692 | 87692 | 87692 | 2327.91 | 21 | 0.32 | 2.35(2.25) | 0.49 | 2.43(2.55) |
| 88184 | 88184 | | 2337.08 | 35.66 | 0.62 | 2.87(2.89) | 0.26 | 2.37(2.41) |
| 88622 | 88622 | | 2343.13 | 19.46 | 0.52 | 3.24(3.30) | 0.24 | 2.58(2.60) |
| 91855 | 91855 | | 2414.15 | 19.57 | 0.56 | 3.29(3.37) | 0.26 | 2.71(2.68) |
| 98089 | | | 2559.18 | 19.41 | 0.75 | 3.94(4.05) | 0.52 | 3.25(3.31) |
| 98720 | 98720 | 98720 | 2565.14 | 23.74 | 0.48 | 2.56(2.60) | 0.29 | 2.12(2.08) |
| 106195 | | | 2716.36 | 20.19 | 0.48 | 3.72(3.39) | 0.09 | 3.39(2.97) |
| 107360 | 107360 | 107360 | 2740.23 | 23.46 | 0.35 | 2.79(2.92) | 0.1 | 2.90(2.99) |
| 107452 | | | 2742.25 | 42.14 | 0.36 | 2.12(2.22) | 0.73 | 2.59(2.57) |
| 107571 | | | 2744.13 | 35.11 | 0.14 | 1.63(1.47) | 0.60 | 2.03(2.10) |
| 107813 | 107813 | | 2750.3 | 28.32 | 0.6 | 2.70(2.80) | 0.39 | 2.32(2.29) |
| 108327 | | | 2761.31 | 21.49 | 0.79 | 3.46(3.44) | 0.59 | 3.01(2.98) |
| 109937 | 109937 | 109937 | 2796.24 | 28.56 | 0.62 | 2.56(2.52) | 0.26 | 2.18(2.18) |
| 110841 | 110841 | 110841 | 2821.32 | 23.71 | 0.49 | 2.60(2.73) | 0.18 | 2.28(2.46) |
| 111304 | 111304 | | 2834.19 | 22.47 | 0.19 | 2.05(1.98) | 0.52 | 2.26(2.25) |
| 111426 | | | 2837.36 | 23.87 | 0.6 | 3.03(3.16) | 0.39 | 2.55(2.45) |
| 111863 | | | 2849.27 | 23.34 | 0.07 | 2.31(2.13) | 0.49 | 2.30(2.31) |
| 112013 | 112013 | 112013 | 2853.33 | 23.78 | 0.63 | 3.09(3.10) | 0.32 | 2.55(2.49) |
| 112106 | | | 2854.36 | 34.86 | 0.75 | 3.12(3.23) | 0.96 | 3.45(3.56) |
| 112839 | 112839 | 112839 | 2873.33 | 28.56 | 0.29 | 2.36(2.32) | 0.03 | 1.84(1.84) |
| 114207 | 114207 | 114207 | 2911.31 | 21.66 | 0.27 | 2.32(2.04) | 0.13 | 2.09(2.26) |
| 114230 | | | 2912.17 | 25.56 | 0.83 | 2.72(2.80) | 0.96 | 3.00(3.04) |
| 116812 | | | 2977.18 | 19.52 | 0.21 | 2.48(2.60) | 0.62 | 2.61(2.65) |
| 117770 | | | 3001.43 | 35.4 | 0.65 | 3.36(3.27) | 0.97 | 3.98(4.11) |
| 118224 | | | 3013.29 | 22.29 | 0.81 | 3.42(3.47) | 0.95 | 3.71(3.75) |
| 118597 | | | 3021.35 | 23.42 | 0.54 | 2.78(2.90) | 0.86 | 3.07(3.14) |
| 118694 | 118694 | | 3023.36 | 24.56 | 0.59 | 2.89(3.08) | 0.35 | 2.17(2.21) |
| 119292 | | | 3035.19 | 42.02 | 0.27 | 2.21(2.07) | 0.71 | 2.71(2.79) |
| 121070 | | | 3076.23 | 19.58 | 0.30 | 2.21(2.30) | 0.63 | 2.73(2.82) |
| 121716 | 121716 | | 3091.44 | 28.4 | 0.21 | 2.44(2.48) | 0.58 | 2.61(2.65) |
| 124688 | 124688 | | 3185.47 | 25.47 | 0.68 | 2.86(2.93) | 0.4 | 2.22(2.22) |
| 125263 | | | 3205.27 | 19.66 | 0.57 | 2.61(2.68) | 0.82 | 2.88(2.96) |
| 125797 | 125797 | 125797 | 3223.39 | 24.76 | 0.35 | 2.43(2.53) | 0.11 | 2.47(2.36) |
| 125799 | | | 3223.42 | 39.13 | 0.52 | 2.79(2.87) | 0.79 | 2.86(2.91) |
| 128249 | 128249 | 128249 | 3290.5 | 24.14 | 0.4 | 2.59(2.56) | 0.6 | 2.81(2.88) |
| 128329 | | | 3292.54 | 39.42 | 0.68 | 3.42(3.53) | 0.96 | 3.87(3.97) |
| 131990 | | | 3400.30 | 42.07 | 0.23 | 2.09(2.05) | 0.59 | 2.42(2.54) |
| 132980 | | | 3426.31 | 27.70 | 0.16 | 1.88(2.21) | 0.54 | 2.23(2.32) |
| 135412 | 135412 | 135412 | 3510.6 | 40.24 | 0.13 | 2.26(2.09) | 0.51 | 2.33(2.34) |
| 136790 | | | 3559.71 | 24.92 | 0.41 | 2.53(2.66) | 0.76 | 2.72(2.77) |
| 137922 | | | 3583.64 | 41.47 | 0.07 | 2.64(2.67) | 0.38 | 2.30(2.40) |
| 140112 | 140112 | 140112 | 3657.67 | 40.71 | 0.29 | 2.78(2.97) | 0.84 | 3.05(3.19) |
| 143106 | | | 3765.45 | 42.57 | 0.07 | 1.77(1.86) | 0.47 | 2.30(2.31) |
| 145865 | 145865 | 145865 | 3890.78 | 24.39 | 0.68 | 3.26(3.43) | 0.47 | 2.49(2.38) |
| 146151 | 146151 | 146151 | 3906.76 | 24.26 | 0.57 | 3.05(3.25) | 0.2 | 2.57(2.44) |
| 147541 | 147541 | 147541 | 3968.6 | 21.09 | 0.63 | 2.98(3.03) | 0.93 | 3.19(3.24) |
| 150909 | 150909 | 150909 | 4082.94 | 21.09 | 0.29 | 3.53(3.56) | 0.06 | 3.99(3.96) |
| 153795 | | | 4195.12 | 21.40 | 0.48 | 3.11(3.07) | 0.05 | 2.50(2.09) |
| 156450 | | | 4305.97 | 25.15 | 0.41 | 3.10(3.24) | 0.08 | 3.18(3.11) |
| 156878 | 156878 | | 4321.94 | 25.2 | 0.59 | 3.66(3.96) | 0.29 | 2.90(2.71) |
| 159259 | 159259 | | 4404.84 | 20.67 | 0.49 | 2.80(2.83) | 0.74 | 2.93(2.97) |
| 168079 | 168079 | 168079 | 4787.06 | 22.45 | 0.43 | 2.54(2.70) | 0.19 | 2.27(2.28) |
| 175081 | | | 5527.35 | 27.46 | 0.07 | 2.61(2.52) | 0.40 | 2.67(2.66) |
| 181591 | 181591 | 181591 | 8110.83 | 19.82 | 0.16 | 3.31(3.17) | 0.04 | 2.45(2.57) |
| 184206 | 184206 | | 8917.25 | 22.55 | 0.29 | 2.37(2.24) | 0.59 | 2.49(2.51) |
| 189663 | 189663 | 189663 | 12716.79 | 25.9 | 0.11 | 2.24(2.23) | 0.28 | 2.59(2.49) |

In one embodiment, the markers are selected from the following markers of Table 2a:

8503, 13746, 15776, 15800, 16854, 18939, 19773, 20334, 23628, 24393, 25866, 26431, 28103, 28306, 29906, 31480, 32470, 33727, 33840, 33973, 36156, 37056, 37949, 40091, 41514, 42304, 42404, 42833, 45980, 46338, 46649, 48093, 48580, 49958, 50212, 50638, 50904, 51804, 52189, 54687, 58880, 59928, 60259, 61221, 61332, 61984, 64899, 64905, 65746, 69769, 69979, 70413, 72161, 72317, 73434, 73697, 74420, 75025, 76839, 78111, 79720, 81263, 84302, 86426, 87411, 87692, 88184, 88622, 91855, 98089, 98720, 107360, 107813, 108327, 109937, 110841, 111304, 111426, 112013, 112106, 112839, 114207, 114230, 117770, 118224, 118597, 118694, 121716, 124688, 125263, 125797, 125799, 128249, 135412, 136790, 140112, 145865, 146151, 147541, 150909, 156878, 159259, 168079, 181591, 184206, 189663.

TABLE 2b
List of markers that enable the differentiation of a primary sclerosing
cholangitis (PSC) from a cholangiocellular carcinoma (CCC) in a bile sample,
especially in an unclear bile duct stenosis, in a multimarker model.

| | | | | | CCC | | PSC | |
|---|---|---|---|---|---|---|---|---|
| | | more | Mass | CE time | | mean log amp | | mean log amp |
| Protein ID | preferably | preferably | [Da] | [min] | Frequency | (log median) | Frequency | (log median) |
| 176 | | | 836.47 | 27.62 | 0.15 | 1.49(1.61) | 0.38 | 1.87(1.80) |
| 231 | | | 844.48 | 29.48 | 0.23 | 1.86(1.91) | 0.47 | 1.80(1.77) |
| 272 | | | 850.44 | 30.1 | 0.08 | 2.54(2.57) | 0.26 | 2.15(2.57) |
| 290 | 290 | 290 | 852.48 | 21.95 | 0.83 | 2.99(3.02) | 0.92 | 3.43(3.64) |
| 754 | 754 | | 900.53 | 30.27 | 0.36 | 1.51(1.49) | 0.59 | 2.10(2.25) |
| 988 | 988 | 988 | 919.43 | 29.9 | 0.05 | 1.75(1.75) | 0.32 | 1.97(1.94) |
| 1134 | | | 930.55 | 23.13 | 0.46 | 2.13(2.16) | 0.26 | 1.79(2.00) |
| 1223 | | | 937.53 | 27.08 | 0.18 | 1.88(1.68) | 0.38 | 2.09(2.07) |
| 1261 | | | 939.51 | 31.09 | 0.38 | 2.32(2.36) | 0.62 | 2.54(2.52) |
| 1387 | | | 948.58 | 22.02 | 0.33 | 1.81(1.79) | 0.12 | 2.02(1.92) |
| 1711 | | | 970.59 | 23.06 | 0.05 | 1.81(1.81) | 0.24 | 1.72(1.73) |
| 1891 | | | 983.58 | 31.9 | 0.46 | 2.09(2.17) | 0.61 | 2.65(2.66) |
| 2651 | 2651 | | 1038.61 | 23.39 | 0.39 | 2.50(2.42) | 0.61 | 2.63(2.74) |
| 2674 | 2674 | 2674 | 1040.59 | 25.26 | 0.46 | 2.40(2.31) | 0.71 | 2.49(2.53) |
| 2809 | 2809 | 2809 | 1049.59 | 31.98 | 0.31 | 1.95(2.16) | 0.62 | 2.61(2.68) |
| 2832 | | | 1050.75 | 19.55 | 0.23 | 2.45(2.77) | 0.03 | 1.84(1.84) |
| 3085 | 3085 | 3085 | 1070.6 | 31.93 | 0.41 | 2.59(2.66) | 0.61 | 2.53(2.48) |
| 3099 | 3099 | | 1071.62 | 33.07 | 0.41 | 1.67(1.93) | 0.56 | 2.28(2.26) |
| 3169 | 3169 | 3169 | 1076.59 | 32.49 | 0.1 | 2.11(2.16) | 0.36 | 2.63(2.70) |
| 3456 | 3456 | 3456 | 1094.58 | 32.22 | 0.41 | 2.41(2.30) | 0.09 | 1.68(1.61) |
| 3700 | 3700 | 3700 | 1112.59 | 33.47 | 0.49 | 2.77(2.89) | 0.36 | 2.47(2.49) |
| 4065 | 4065 | | 1142.64 | 33.34 | 0.23 | 2.08(2.20) | 0.47 | 2.21(2.40) |
| 4139 | 4139 | | 1148.65 | 33.44 | 0.41 | 2.20(2.23) | 0.62 | 2.66(2.97) |
| 4262 | 4262 | 4262 | 1159.64 | 33.64 | 0.13 | 2.08(1.81) | 0.38 | 2.65(2.75) |
| 4295 | 4613 | 4613 | 1163.61 | 26.14 | 0.21 | 2.26(2.32) | 0.03 | 2.17(2.17) |
| 4720 | 4720 | 4720 | 1200.7 | 24.98 | 0.15 | 2.22(2.13) | 0.25 | 3.34(3.29) |
| 4731 | 4731 | | 1201.66 | 33.84 | 0.21 | 2.18(2.01) | 0.5 | 2.33(2.40) |
| 4755 | | | 1204.64 | 33.38 | 0.23 | 2.18(2.52) | 0.44 | 2.71(2.62) |
| 4840 | 4840 | | 1212.68 | 33.51 | 0.44 | 2.72(2.87) | 0.67 | 2.87(2.91) |
| 4909 | 4909 | | 1222.67 | 33.6 | 0.41 | 2.47(2.50) | 0.65 | 2.67(2.75) |
| 4995 | 4995 | 4995 | 1231.63 | 34.07 | 0.54 | 2.68(2.79) | 0.71 | 3.11(3.14) |
| 5086 | 5086 | | 1242.67 | 33.64 | 0.28 | 2.34(2.81) | 0.53 | 2.60(2.54) |
| 5103 | 5103 | 5103 | 1243.67 | 33.55 | 0.34 | 2.86(2.82) | 0.58 | 2.68(2.73) |
| 5131 | 5131 | 5131 | 1246.67 | 33.57 | 0.1 | 2.87(2.99) | 0.38 | 3.06(3.35) |
| 5230 | | | 1256.67 | 25.21 | 0.08 | 1.60(1.39) | 0.26 | 2.19(2.39) |
| 5328 | | | 1266.72 | 25.46 | 0.31 | 2.42(2.45) | 0.12 | 2.05(2.07) |
| 5448 | 5448 | | 1278.79 | 35 | 0.08 | 2.67(2.35) | 0.32 | 2.61(2.65) |
| 5510 | 5510 | 5510 | 1284.69 | 33.83 | 0.18 | 2.04(1.86) | 0.38 | 2.78(2.45) |
| 5741 | 5741 | | 1309.71 | 34.13 | 0.36 | 2.04(1.99) | 0.56 | 2.74(2.74) |
| 5775 | 5775 | | 1313.71 | 34.24 | 0.38 | 2.20(2.22) | 0.59 | 2.54(2.56) |
| 5787 | | | 1314.75 | 25.79 | 0.33 | 2.22(2.27) | 0.09 | 1.78(1.91) |
| 5851 | | | 1322.7 | 33.89 | 0.1 | 2.49(2.30) | 0.29 | 2.48(2.42) |
| 6054 | 6054 | 6054 | 1344.76 | 31.13 | 0.24 | 2.29(2.04) | 0.22 | 2.36(2.36) |
| 6072 | | | 1346.78 | 28.82 | 0.13 | 1.91(2.11) | 0.32 | 2.26(2.12) |
| 6135 | 6135 | | 1354.72 | 25.47 | 0.28 | 2.38(2.34) | 0.53 | 2.56(2.65) |
| 6174 | | | 1358.72 | 34.27 | 0.26 | 2.83(3.12) | 0.53 | 2.88(2.89) |
| 6270 | | | 1370.7 | 34.14 | 0.13 | 2.44(2.92) | 0.35 | 2.29(2.43) |
| 6366 | 6366 | 6366 | 1380.78 | 26.56 | 0.41 | 2.89(2.77) | 0.62 | 3.07(3.10) |
| 6436 | 6436 | 6436 | 1388.76 | 26.84 | 0.64 | 3.28(3.33) | 0.47 | 2.68(2.77) |
| 6438 | 6438 | | 1388.74 | 34.52 | 0.31 | 2.59(2.77) | 0.5 | 3.19(3.34) |
| 6507 | 6507 | 6507 | 1397.72 | 34.85 | 0.37 | 1.89(1.67) | 0.22 | 1.95(1.87) |
| 6885 | 6885 | | 1440.78 | 29.43 | 0.27 | 2.60(2.39) | 0.53 | 2.56(2.50) |
| 7067 | | | 1462.76 | 34.56 | 0.1 | 1.89(1.90) | 0.29 | 2.32(2.38) |
| 7266 | | | 1482.88 | 35.1 | 0.36 | 2.82(2.63) | 0.53 | 3.72(3.63) |
| 7475 | 7475 | 7475 | 1508.82 | 27.62 | 0.44 | 2.16(2.13) | 0.12 | 2.57(2.57) |
| 7639 | 7639 | 7639 | 1528.82 | 35.05 | 0.62 | 3.35(3.46) | 0.85 | 3.57(3.62) |
| 7723 | | | 1539.84 | 35.21 | 0.23 | 2.91(2.90) | 0.47 | 2.63(2.72) |
| 7785 | 7785 | 7785 | 1547.81 | 35.18 | 0.39 | 2.73(2.63) | 0.19 | 2.34(2.03) |
| 8084 | 8084 | 8084 | 1582.81 | 35.15 | 0.13 | 1.76(1.85) | 0.35 | 2.21(2.04) |
| 8112 | 8112 | | 1587.82 | 28.34 | 0.33 | 2.32(2.28) | 0.59 | 2.57(2.53) |
| 8386 | 8386 | 8386 | 1619.84 | 35.25 | 0.29 | 2.22(2.19) | 0.25 | 2.27(2.12) |
| 8518 | | | 1636 | 20.67 | 0.36 | 2.69(2.90) | 0.15 | 2.26(2.69) |
| 8621 | | | 1646.88 | 35.67 | 0.46 | 2.42(2.53) | 0.24 | 2.45(2.53) |
| 8662 | 8662 | 8662 | 1651.86 | 35.51 | 0.46 | 2.69(2.33) | 0.12 | 2.05(2.22) |
| 8727 | 8727 | | 1659.87 | 28.3 | 0.33 | 2.83(2.92) | 0.56 | 2.75(3.02) |
| 8959 | | | 1687.87 | 28.53 | 0.21 | 2.36(2.29) | 0.38 | 2.65(2.63) |
| 9029 | | | 1696.91 | 28.63 | 0.1 | 1.70(1.92) | 0.29 | 2.41(2.12) |
| 9032 | 9032 | 9032 | 1696.91 | 35.65 | 0.33 | 2.30(2.28) | 0.12 | 1.91(2.12) |
| 9086 | 9086 | | 1704.9 | 28.58 | 0.56 | 3.49(3.57) | 0.68 | 3.98(4.00) |
| 9106 | | | 1707.01 | 23.73 | 0.28 | 2.67(2.53) | 0.12 | 1.85(2.00) |
| 9151 | 9151 | 9151 | 1712.93 | 35.61 | 0.51 | 3.26(3.45) | 0.76 | 3.35(3.43) |
| 9528 | 9528 | 9528 | 1759.94 | 35.72 | 0.33 | 2.23(2.18) | 0.06 | 2.66(2.66) |
| 9554 | | | 1762.97 | 36.12 | 0.08 | 3.30(3.16) | 0.26 | 2.67(2.56) |
| 9602 | | | 1769.91 | 35.87 | 0.33 | 2.68(2.59) | 0.15 | 1.95(2.14) |
| 9944 | 9944 | 9944 | 1812.07 | 24.12 | 0.36 | 3.12(3.30) | 0.09 | 2.18(1.87) |
| 10357 | | | 1867.96 | 36.19 | 0.36 | 2.05(2.23) | 0.15 | 2.10(1.75) |
| 10784 | 10784 | 10784 | 1926.99 | 36.49 | 0.46 | 2.19(2.13) | 0.25 | 2.30(2.47) |
| 10893 | 10893 | | 1944.03 | 23.99 | 0.23 | 3.01(2.94) | 0.5 | 3.24(2.98) |
| 10984 | 10984 | | 1956.09 | 24.72 | 0.26 | 3.94(4.26) | 0.03 | 2.73(2.73) |
| 11096 | 11096 | 11096 | 1972.08 | 25.48 | 0.41 | 2.43(2.25) | 0.12 | 2.44(2.62) |
| 11283 | | | 2002.15 | 25.43 | 0.08 | 1.68(2.08) | 0.26 | 2.59(2.61) |
| 11334 | | | 2009.07 | 31.13 | 0.03 | 3.21(3.21) | 0.21 | 2.72(2.90) |
| 11467 | 11467 | | 2027.13 | 35.4 | 0.21 | 2.36(2.23) | 0.44 | 2.62(2.67) |
| 11501 | | | 2032.1 | 37.12 | 0.13 | 1.96(2.00) | 0.32 | 1.89(2.01) |
| 12098 | 12098 | 12098 | 2125.21 | 31.46 | 0.21 | 2.13(1.97) | 0.5 | 2.96(3.25) |
| 12534 | | | 2184.13 | 37.02 | 0.18 | 1.57(1.61) | 0.35 | 2.19(2.02) |
| 12746 | 12746 | 12746 | 2215.15 | 32.1 | 0.08 | 1.87(2.01) | 0.41 | 2.61(2.79) |
| 12829 | | | 2226.23 | 32.28 | 0.26 | 2.93(2.81) | 0.5 | 2.84(2.96) |
| 13065 | | | 2260.21 | 23.11 | 0.36 | 2.67(2.52) | 0.15 | 2.74(2.81) |
| 13807 | 13807 | | 2371.23 | 32.81 | 0.03 | 1.95(1.95) | 0.24 | 2.28(2.09) |
| 14143 | 14143 | 14143 | 2423.28 | 36.64 | 0.1 | 1.98(2.12) | 0.38 | 2.18(1.95) |
| 15246 | | | 2596.36 | 28.27 | 0.08 | 2.08(1.90) | 0.26 | 1.91(1.99) |
| 15375 | | | 2616.37 | 37.92 | 0.17 | 1.53(1.47) | 0 | 0.00(0.00) |
| 15571 | | | 2650.41 | 33.63 | 0.18 | 1.85(1.57) | 0.41 | 1.97(1.96) |
| 16759 | 16759 | 16759 | 2867.54 | 30.89 | 0.27 | 2.70(2.95) | 0.25 | 2.62(2.59) |
| 17000 | 17000 | 17000 | 2917.53 | 38.5 | 0.08 | 1.71(1.65) | 0.35 | 2.10(1.87) |
| 17831 | | | 3087.71 | 29.81 | 0.05 | 1.22(1.22) | 0.21 | 2.08(1.90) |
| 18203 | | | 3166.71 | 26.1 | 0.05 | 3.03(3.03) | 0.21 | 3.07(3.59) |
| 19487 | 19487 | 19487 | 3468.01 | 28.42 | 0.07 | 2.01(2.03) | 0.22 | 2.22(2.12) |
| 22973 | | | 4960.56 | 22.91 | 0.21 | 3.81(3.63) | 0.03 | 3.55(3.55) |
| 23004 | | | 4976.57 | 23.1 | 0.21 | 4.59(4.65) | 0.03 | 2.52(2.52) |
| 23618 | | | 5574.38 | 27.48 | 0.28 | 3.46(3.28) | 0.06 | 2.90(2.90) |
| 24144 | 24144 | 24144 | 6236.96 | 23.77 | 0.44 | 4.15(3.77) | 0.15 | 3.40(3.25) |
| 149713 | | | 806.47 | 21 | 0.1 | 1.44(1.56) | 0.26 | 1.97(1.94) |
| 149721 | 149721 | | 811.38 | 39.4 | 0.03 | 2.14(2.14) | 0.24 | 1.84(2.05) |
| 149753 | | | 838.49 | 22.35 | 0.15 | 1.42(1.34) | 0.38 | 1.53(1.59) |
| 149768 | | | 847.45 | 29.8 | 0.26 | 1.44(1.14) | 0.44 | 1.97(1.98) |
| 149778 | | | 854.41 | 40.06 | 0.03 | 2.17(2.17) | 0.21 | 1.92(1.96) |
| 149779 | | | 854.48 | 29.59 | 0.1 | 2.16(2.19) | 0.35 | 1.38(1.52) |
| 149781 | 149781 | | 855.5 | 30.41 | 0.03 | 0.68(0.68) | 0.24 | 1.77(1.70) |
| 149810 | | | 879.41 | 38.89 | 0.03 | 1.01(1.01) | 0.21 | 1.67(1.78) |
| 149815 | | | 880.45 | 28.99 | 0.13 | 2.01(2.03) | 0.35 | 1.92(1.72) |
| 149823 | 149823 | | 884.47 | 27.77 | 0.03 | 1.16(1.16) | 0.29 | 1.44(1.66) |
| 149845 | | | 895.46 | 32.22 | 0.15 | 1.25(1.16) | 0.35 | 1.53(1.43) |
| 149854 | | | 900.54 | 22.61 | 0.13 | 2.27(2.42) | 0.35 | 2.39(2.67) |
| 149864 | | | 907.51 | 22.66 | 0.05 | 1.91(1.91) | 0.26 | 1.91(2.39) |
| 149873 | | | 912.51 | 32.38 | 0.15 | 1.83(1.64) | 0.41 | 1.73(1.61) |
| 149885 | 149885 | | 920.46 | 39.93 | 0 | 0.00(0.00) | 0.21 | 1.69(1.72) |
| 149887 | | | 921.48 | 30.25 | 0.31 | 2.19(2.25) | 0.53 | 2.48(2.42) |
| 149895 | | | 926.49 | 34.35 | 0.21 | 1.24(1.17) | 0.03 | 1.06(1.06) |
| 149914 | | | 940.53 | 28.82 | 0.1 | 1.64(1.65) | 0.29 | 1.76(1.61) |
| 149916 | 149916 | 149916 | 941.52 | 30.36 | 0.1 | 2.04(2.20) | 0.38 | 2.38(2.41) |
| 149944 | 149944 | 149944 | 967.53 | 30 | 0.05 | 2.23(2.23) | 0.32 | 2.37(2.40) |
| 149959 | | | 974.59 | 24.3 | 0.26 | 2.29(2.22) | 0.09 | 1.61(1.35) |
| 149962 | | | 981.48 | 42.56 | 0.26 | 1.74(1.79) | 0.09 | 0.88(0.96) |
| 149982 | | | 990.58 | 24.84 | 0.08 | 1.40(1.49) | 0.29 | 1.84(1.97) |
| 149990 | 149990 | | 999.64 | 31.72 | 0.03 | 3.02(3.02) | 0.26 | 3.25(3.28) |
| 150006 | | | 1011.58 | 23.61 | 0.1 | 2.08(2.15) | 0.29 | 2.46(2.60) |
| 150017 | | | 1019.53 | 30.93 | 0.28 | 2.27(2.33) | 0.5 | 2.58(2.57) |
| 150068 | | | 1051.57 | 32.41 | 0.1 | 2.21(2.23) | 0.35 | 2.32(2.56) |
| 150071 | | | 1054.58 | 32.64 | 0.15 | 2.09(1.79) | 0.38 | 1.95(1.76) |
| 150087 | | | 1066.58 | 31.77 | 0.1 | 2.55(3.06) | 0.29 | 2.25(2.29) |
| 150091 | | | 1068.64 | 23.76 | 0.28 | 1.81(1.65) | 0.09 | 1.94(1.95) |
| 150109 | | | 1086.51 | 43.23 | 0.26 | 1.51(1.64) | 0.06 | 1.47(1.47) |
| 150139 | 150139 | | 1111.64 | 26.57 | 0.03 | 1.71(1.71) | 0.24 | 1.78(1.64) |
| 150158 | | | 1127.62 | 33.56 | 0.08 | 1.75(1.67) | 0.29 | 1.79(1.64) |
| 150159 | | | 1127.74 | 24.66 | 0.05 | 3.64(3.64) | 0.24 | 3.22(3.21) |
| 150166 | 150166 | | 1133.61 | 33.28 | 0.36 | 2.27(2.47) | 0.12 | 1.26(0.98) |
| 150175 | | | 1139.64 | 41.79 | 0.05 | 1.96(1.96) | 0.24 | 2.01(1.78) |
| 150182 | | | 1147.66 | 21.71 | 0.23 | 1.70(1.70) | 0.06 | 1.56(1.56) |
| 150185 | | | 1149.75 | 35.14 | 0.05 | 2.76(2.76) | 0.26 | 2.65(2.55) |
| 150190 | | | 1155.62 | 41.79 | 0.13 | 2.13(1.66) | 0.35 | 2.58(2.49) |
| 150198 | 150198 | 150198 | 1167.72 | 33.32 | 0.08 | 2.68(2.42) | 0.35 | 3.00(3.19) |
| 150201 | | | 1168.69 | 26.5 | 0.23 | 2.73(2.75) | 0.06 | 1.67(1.67) |
| 150205 | | | 1174.66 | 26.79 | 0.23 | 1.95(1.67) | 0.06 | 1.75(1.75) |
| 150235 | | | 1202.69 | 25.98 | 0.21 | 2.33(2.44) | 0.03 | 3.06(3.06) |
| 150289 | | | 1272.69 | 22.08 | 0.05 | 1.87(1.87) | 0.24 | 1.90(1.82) |
| 150310 | 150310 | | 1293.68 | 28.56 | 0.03 | 3.11(3.11) | 0.26 | 2.31(2.45) |
| 150314 | | | 1298.69 | 34.06 | 0.21 | 2.13(2.16) | 0.03 | 1.75(1.75) |
| 150320 | 150320 | | 1302.74 | 25.77 | 0.03 | 2.41(2.41) | 0.24 | 2.83(2.77) |
| 150343 | | | 1327.71 | 34.24 | 0.41 | 2.88(2.78) | 0.68 | 2.77(2.82) |
| 150380 | | | 1369.7 | 34.22 | 0.31 | 2.90(3.15) | 0.12 | 1.86(2.17) |
| 150394 | | | 1383.81 | 23.64 | 0.23 | 1.90(1.76) | 0.06 | 1.58(1.58) |
| 150425 | | | 1413.79 | 21.57 | 0.05 | 2.20(2.20) | 0.24 | 2.00(2.08) |
| 150429 | | | 1415.94 | 21.82 | 0.21 | 3.24(3.38) | 0.03 | 1.75(1.75) |
| 150450 | | | 1429.74 | 34.42 | 0.33 | 2.73(3.06) | 0.12 | 2.37(2.47) |
| 150453 | | | 1431.69 | 43.82 | 0.1 | 2.61(2.60) | 0.29 | 3.00(3.04) |
| 150460 | | | 1437.83 | 27.23 | 0.26 | 2.20(2.22) | 0.06 | 1.15(1.15) |
| 150490 | | | 1469.65 | 44.36 | 0.1 | 3.19(3.38) | 0.32 | 3.15(3.59) |
| 150500 | | | 1478.77 | 34.68 | 0.31 | 2.23(1.95) | 0.12 | 1.86(1.82) |
| 150518 | | | 1491.81 | 27.3 | 0.05 | 1.36(1.36) | 0.21 | 1.55(1.26) |
| 150540 | | | 1513.78 | 34.83 | 0.28 | 2.49(2.47) | 0.09 | 2.69(2.73) |
| 150548 | | | 1524.81 | 35.28 | 0.13 | 2.34(2.41) | 0.35 | 2.01(2.04) |
| 150549 | | | 1524.84 | 28.38 | 0.26 | 2.55(2.45) | 0.06 | 1.89(1.89) |
| 150550 | | | 1526.9 | 22.5 | 0.23 | 2.86(2.98) | 0.06 | 1.39(1.39) |
| 150571 | | | 1554.89 | 22.84 | 0.26 | 2.31(2.39) | 0.09 | 1.39(1.42) |
| 150574 | | | 1556.92 | 22.57 | 0.1 | 1.96(1.74) | 0.29 | 2.09(2.22) |
| 150579 | | | 1559.83 | 28.51 | 0.08 | 1.79(1.51) | 0.26 | 2.14(2.14) |
| 150633 | 150633 | | 1605.87 | 22.89 | 0.21 | 1.96(1.82) | 0 | 0.00(0.00) |
| 150664 | | | 1627.95 | 23.12 | 0.28 | 2.80(2.73) | 0.06 | 2.40(2.40) |
| 150678 | | | 1651.93 | 28.55 | 0.13 | 2.11(2.30) | 0.32 | 2.23(2.26) |
| 150694 | | | 1671.98 | 28.74 | 0.23 | 2.06(2.30) | 0.06 | 2.28(2.28) |
| 150714 | | | 1688.91 | 29.07 | 0.26 | 3.38(3.28) | 0.06 | 2.46(2.46) |
| 150728 | 150728 | | 1699.92 | 35.81 | 0.33 | 2.92(2.98) | 0.09 | 2.16(2.26) |
| 150733 | | | 1705.98 | 23.55 | 0.05 | 2.06(2.06) | 0.21 | 2.04(1.71) |
| 150744 | 150744 | | 1714.94 | 23.49 | 0.26 | 2.41(2.12) | 0.03 | 2.85(2.85) |
| 150758 | | | 1724.92 | 29.38 | 0.21 | 2.96(2.73) | 0.03 | 2.00(2.00) |
| 150775 | | | 1740.94 | 29.67 | 0.21 | 2.45(2.57) | 0.03 | 1.81(1.81) |
| 150778 | 150778 | | 1748.9 | 35.28 | 0.21 | 2.26(2.38) | 0 | 0.00(0.00) |
| 150799 | | | 1765.94 | 35.61 | 0.1 | 1.22(1.20) | 0.26 | 2.30(2.37) |
| 150812 | | | 1779.02 | 24.01 | 0.21 | 2.22(2.29) | 0.41 | 2.61(2.54) |
| 150817 | | | 1782.99 | 30.91 | 0.1 | 2.38(2.50) | 0.35 | 2.39(2.30) |
| 150828 | | | 1796.92 | 35.6 | 0.23 | 1.84(2.09) | 0.06 | 2.01(2.01) |
| 150850 | | | 1811.98 | 36.55 | 0.1 | 2.43(2.65) | 0.29 | 2.52(2.61) |
| 150893 | | | 1849 | 24.68 | 0.21 | 1.92(1.90) | 0.03 | 0.70(0.70) |
| 150898 | | | 1852.95 | 36.26 | 0.33 | 2.60(2.86) | 0.12 | 1.82(1.80) |
| 150900 | 150900 | | 1853.99 | 29.86 | 0.26 | 2.82(2.91) | 0.03 | 1.32(1.32) |
| 150942 | 150942 | | 1899.03 | 30.75 | 0.26 | 2.19(1.97) | 0.03 | 0.82(0.82) |
| 150958 | 150958 | | 1913.04 | 35.41 | 0.05 | 1.78(1.78) | 0.29 | 1.90(1.78) |
| 151007 | | | 1960.08 | 24.65 | 0.03 | 0.79(0.79) | 0.21 | 2.07(2.46) |
| 151008 | | | 1961.09 | 22.06 | 0.05 | 1.47(1.47) | 0.24 | 2.57(2.88) |
| 151015 | | | 1966.14 | 25.06 | 0.05 | 2.42(2.42) | 0.21 | 2.42(2.50) |
| 151033 | | | 1986.06 | 31.31 | 0.23 | 3.57(3.88) | 0.03 | 1.46(1.46) |
| 151044 | 151044 | | 2003.18 | 24.16 | 0.03 | 2.64(2.64) | 0.24 | 1.93(2.06) |
| 151052 | | | 2015.1 | 36.27 | 0.03 | 2.03(2.03) | 0.21 | 1.61(1.57) |
| 151061 | | | 2024.08 | 36.98 | 0.05 | 1.46(1.46) | 0.24 | 1.64(1.56) |
| 151069 | | | 2029.09 | 30.65 | 0.23 | 2.36(2.88) | 0.06 | 2.69(2.69) |
| 151071 | | | 2031.08 | 25.09 | 0.33 | 2.00(1.61) | 0.12 | 2.37(2.59) |
| 151094 | | | 2056.03 | 36.55 | 0.05 | 2.43(2.43) | 0.24 | 1.83(1.73) |
| 151097 | | | 2058.13 | 31.81 | 0.23 | 2.22(2.33) | 0.03 | 2.81(2.81) |
| 151098 | | | 2059.06 | 36.26 | 0.03 | 0.97(0.97) | 0.21 | 1.79(1.54) |
| 151122 | | | 2079.95 | 30.14 | 0.13 | 2.72(2.67) | 0.32 | 3.22(3.16) |
| 151125 | | | 2081.08 | 36.88 | 0.23 | 2.19(2.22) | 0.03 | 2.55(2.55) |
| 151129 | | | 2088.29 | 22.36 | 0.21 | 3.34(3.75) | 0.03 | 2.33(2.33) |
| 151142 | | | 2108.14 | 26.07 | 0.03 | 2.10(2.10) | 0.21 | 1.97(1.79) |
| 151150 | 151150 | | 2116.16 | 25.76 | 0.05 | 1.46(1.46) | 0.26 | 2.25(2.22) |
| 151166 | | | 2133.1 | 36.95 | 0.05 | 1.72(1.72) | 0.21 | 2.21(2.38) |
| 151176 | | | 2145.17 | 25.92 | 0.05 | 2.58(2.58) | 0.21 | 1.82(1.85) |
| 151200 | 151200 | | 2171.22 | 32.68 | 0.28 | 2.95(2.69) | 0.06 | 1.28(1.28) |
| 151219 | | | 2189.33 | 22.89 | 0.28 | 3.37(3.23) | 0.09 | 2.14(2.15) |
| 151229 | | | 2198.17 | 32.69 | 0.28 | 3.24(3.46) | 0.09 | 1.85(2.08) |
| 151235 | | | 2202.31 | 22.23 | 0.26 | 2.54(2.32) | 0.06 | 2.18(2.18) |
| 151259 | | | 2232.19 | 32.69 | 0.23 | 2.52(3.06) | 0.03 | 2.76(2.76) |
| 151268 | | | 2247.23 | 26.48 | 0.23 | 2.89(3.07) | 0.06 | 1.20(1.20) |
| 151285 | 151285 | | 2272.34 | 29.24 | 0.23 | 2.36(1.88) | 0 | 0.00(0.00) |
| 151291 | | | 2280.21 | 32.64 | 0.21 | 1.70(1.28) | 0.03 | 1.92(1.92) |
| 151315 | | | 2317.26 | 36.47 | 0.03 | 2.07(2.07) | 0.21 | 1.64(1.58) |
| 151320 | | | 2321.29 | 27.38 | 0.03 | 1.07(1.07) | 0.21 | 1.85(2.10) |
| 151353 | | | 2370.25 | 26.74 | 0.08 | 2.27(2.11) | 0.26 | 2.05(2.48) |
| 151360 | 151360 | | 2377.3 | 27.13 | 0.05 | 2.99(2.99) | 0.29 | 2.63(3.05) |
| 151371 | 151371 | | 2393.27 | 33.18 | 0.03 | 2.46(2.46) | 0.24 | 1.28(1.25) |
| 151388 | | | 2416.44 | 23.3 | 0.31 | 2.79(2.76) | 0.09 | 2.66(2.66) |
| 151391 | | | 2421.27 | 33.6 | 0.23 | 2.55(2.72) | 0.06 | 2.29(2.29) |
| 151397 | | | 2434.31 | 33.21 | 0.05 | 1.47(1.47) | 0.24 | 2.12(2.08) |
| 151423 | | | 2487.48 | 23.57 | 0.31 | 2.87(2.77) | 0.12 | 2.70(2.43) |
| 151436 | 151436 | 151436 | 2507.4 | 28.1 | 0.33 | 2.69(2.14) | 0 | 0.00(0.00) |
| 151450 | 151450 | | 2532.42 | 24.15 | 0.21 | 2.22(2.13) | 0 | 0.00(0.00) |
| 151458 | | | 2552.41 | 27.64 | 0.15 | 1.71(1.75) | 0.35 | 2.38(2.51) |
| 151462 | | | 2559.36 | 33.65 | 0.03 | 1.65(1.65) | 0.21 | 2.13(1.80) |
| 151465 | | | 2560.39 | 33.61 | 0.05 | 2.56(2.56) | 0.24 | 1.95(1.80) |
| 151484 | 151484 | | 2596.55 | 21.96 | 0.26 | 3.12(3.18) | 0.03 | 2.28(2.28) |
| 151491 | 151491 | | 2614.38 | 28.34 | 0.23 | 2.91(3.19) | 0 | 0.00(0.00) |
| 151503 | | | 2637.28 | 27.88 | 0.1 | 2.64(2.93) | 0.32 | 3.05(3.02) |
| 151540 | | | 2746.36 | 28.53 | 0.03 | 2.57(2.57) | 0.21 | 2.29(2.11) |
| 151547 | 151547 | 151547 | 2775.52 | 25.23 | 0.31 | 2.27(2.21) | 0.06 | 1.89(1.89) |
| 151573 | | | 2871.52 | 29.41 | 0.03 | 3.35(3.35) | 0.21 | 1.89(1.63) |
| 151580 | | | 2901.57 | 25.12 | 0.03 | 2.20(2.20) | 0.21 | 1.64(1.63) |
| 151586 | | | 2917.55 | 34.51 | 0.23 | 3.27(3.86) | 0.06 | 1.24(1.24) |
| 151593 | | | 2956.61 | 23.07 | 0.36 | 3.06(3.14) | 0.15 | 3.05(3.10) |
| 151594 | | | 2964.63 | 30.1 | 0.05 | 2.58(2.58) | 0.21 | 2.52(2.71) |
| 151640 | 151640 | | 3167.66 | 30.11 | 0 | 0.00(0.00) | 0.26 | 2.63(2.98) |
| 151661 | | | 3327.75 | 35.03 | 0.08 | 2.80(2.61) | 0.26 | 2.27(2.56) |
| 151667 | 151667 | | 3376.83 | 34.68 | 0.05 | 2.40(2.40) | 0.29 | 2.61(2.78) |
| 151685 | | | 3489.67 | 33.76 | 0.03 | 3.16(3.16) | 0.21 | 1.93(1.31) |
| 151688 | 151688 | | 3520.81 | 23.96 | 0.21 | 2.90(2.60) | 0 | 0.00(0.00) |
| 151711 | 151711 | | 4045.28 | 21.47 | 0.21 | 3.49(3.28) | 0 | 0.00(0.00) |
| 151728 | | | 4272.27 | 25.9 | 0.05 | 3.00(3.00) | 0.21 | 2.71(2.71) |
| 151770 | | | 8288.94 | 19.99 | 0.31 | 3.00(2.87) | 0.12 | 3.18(2.82) |
| 151775 | | | 9948.59 | 21.08 | 0.23 | 2.83(2.23) | 0.03 | 2.42(2.42) |
| 151776 | 151776 | | 11721.3 | 20.14 | 0.23 | 3.26(2.75) | 0 | 0.00(0.00) |
| 151777 | 151777 | | 11737.3 | 20.28 | 0.28 | 3.43(3.43) | 0 | 0.00(0.00) |

In one embodiment, the markers are selected from the following markers of Table 2b:

176, 231, 272, 290, 754, 988, 1134, 1223, 1261, 1387, 1711, 2651, 2674, 2809, 2832, 3085, 3099, 3169, 3456, 3700, 4065, 4139, 4262, 4295, 4720, 4731, 4755, 4840, 4909, 4995, 5086, 5103, 5131, 5230, 5328, 5448, 5510, 5741, 5775, 5787, 5851, 6054, 6072, 6135, 6174, 6270, 6366, 6436, 6438, 6507, 6885, 7067, 7266, 7475, 7639, 7723, 7785, 8084, 8112, 8386, 8518, 8621, 8662, 8727, 8959, 9029, 9032, 9086, 9106, 9151, 9528, 9554, 9602, 9944, 10357, 10784, 10893, 10984, 11096, 11283, 11334, 11467, 11501, 12098, 12534, 12746, 12829, 13065, 13807, 14143, 15246, 15571, 16759, 17000, 17831, 18203, 19487, 22973, 23004, 23618, 24144, 149713, 149721, 149753, 149768, 149778, 149779, 149781, 149810, 149815, 149823, 149845, 149854, 149864, 149873, 149885, 149887, 149895, 149914, 149916, 149944, 149959, 149962, 149982, 149990, 150006, 150017, 150068, 150071, 150087, 150091, 150109, 150139, 150158, 150159, 150166, 150175, 150182, 150185, 150190, 150198, 150201, 150205, 150235, 150289, 150310, 150314, 150320, 150343, 150380, 150394, 150425, 150429, 150450, 150453, 150460, 150490, 150500, 150518, 150540, 150548, 150549, 150550, 150571, 150574, 150579, 150633, 150664, 150678, 150694, 150714, 150728, 150733, 150744, 150758, 150775, 150778, 150799, 150812, 150817, 150828, 150850, 150893, 150898, 150900, 150942, 150958, 151007, 151008, 151015, 151033, 151044, 151052, 151061, 151069, 151071, 151094, 151097, 151098, 151122, 151125, 151129, 151142, 151150, 151166, 151176, 151200, 151219, 151229, 151235, 151259, 151268, 151285, 151291, 151315, 151320, 151353, 151360, 151371, 151388, 151391, 151397, 151423, 151436, 151450, 151458, 151462, 151465, 151484, 151491, 151503, 151540, 151547, 151573, 151580, 151586, 151593, 151594, 151640, 151661, 151667, 151685, 151688, 151711, 151728, 151770, 151775, 151776, 151777.

The amino acid sequence of many of the peptides is known. It is shown in Table 3 together with the related precursor protein.

TABLE 3
Amino acid sequence and assignable precursor protein of the markers
with known sequence that are relevant to the recognition and differ-
entiation of a bile duct stenosis.

| Protein | | | Start | Stop | Swissprot |
|---|---|---|---|---|---|
| ID | Sequence | Protein name | AA | AA | name |
| 1826 | SLDKFLASV | Hemoglobin subunit alpha | 125 | 133 | HBA_HUMAN |
| 3099 | IGDEAIEKPT | Actin-related protein 3 | 62 | 71 | ARP3_HUMAN |
| 3252 | VADALTNAVAH | Hemoglobin subunit alpha | 63 | 73 | HBA_HUMAN |
| 4065 | LELAGNAARDN | Histone H2A type 1 | 64 | 74 | H2A1_HUMAN |
| 4139 | LVNEVTEFAK | Serum albumin | 66 | 75 | ALBU_HUMAN |
| 4262 | DLLDDLKSEL | Annexin A5 | 64 | 73 | ANXA5_HUMAN |
| 4840 | WLQGSQELPR | Ig alpha-1 chain C region | 408 | 417 | IGHA1_HUMAN |
| 4847 | IKDPDASKPED | Calreticulin | 208 | 218 | CALR_HUMAN |
| 5086 | DLKSVLGQLGIT | Alpha-l-antitrypsin | 24 | 35 | A1AT_HUMAN |
| 5519 | GKVNVDEVGGEAL | Hemoglobin subunit beta | 17 | 29 | HBB_HUMAN |
| 5775 | VNVDEVGGEALGR | Hemoglobin subunit beta | 19 | 31 | HBB_HUMAN |
| 5902 | FSDGLAHLDNLK | Hemoglobin subunit beta | 72 | 83 | HBB_HUMAN |
| 5936 | KVVATTQMQAADA | Aminopeptidase N | 205 | 217 | AMPN_HUMAN |
| 6012 | SGGTTmYPGIADR | Actin, cytoplasmic 1 | 302 | 314 | ACTB_HUMAN |
| 6054 | FYAPELLFFAK | Serum albumin | 173 | 183 | ALBU_HUMAN |
| 6174 | FQNSAILTIQPK | Complement C5 | 79 | 90 | CO5_HUMAN |
| 6289 | LTEPADTITDAVK | Kallikrein-1 | 126 | 138 | KLK1_HUMAN |
| 6438 | LVTEVENGGSLGSK | Pyruvate kinase isozymes M1/M2 | 193 | 206 | KPYM_HUMAN |
| 6507 | DLAGRDLTDYLm | Beta-actin-like protein 2 | 180 | 191 | ACTBL_HUMAN |
| 7067 | LTGLLDLALEKDY | Pancreatic alpha-amylase | 177 | 189 | AMYP_HUMAN |
| 7101 | TNAVAHVDDMPNAL | Hemoglobin subunit alpha | 68 | 81 | HBA_HUMAN |
| 7143 | DGLAHLDNLKGTFA | Hemoglobin subunit beta | 74 | 87 | HBB_HUMAN |
| 7238 | SLDKFLASVSTVLT | Hemoglobin subunit alpha | 125 | 138 | HBA_HUMAN |
| 7636 | VGAHAGEYGAEALER | Hemoglobin subunit alpha | 18 | 32 | HBA_HUMAN |
| 7639 | RPSGNLVSVLSGAEGS | Regenerating islet-derived | 71 | 86 | REG3A_HUMAN |
| | | protein 3-alpha | | | |
| 7723 | VNVDEVGGEALGRLL | Hemoglobin subunit beta | 19 | 33 | HBB_HUMAN |
| 7874 | SDGLAHLDNLKGTFA | Hemoglobin subunit beta | 73 | 87 | HBB_HUMAN |
| 8182 | VDDMPNALSALSDLH | Hemoglobin subunit alpha | 74 | 88 | HBA_HUMAN |
| 8339 | GAFSDGLAHLDNLKGT | Hemoglobin subunit beta | 70 | 85 | HBB_HUMAN |
| 8386 | SNKYDPPLEDGAMPS | F-actin-capping protein subunit | 76 | 90 | CAPZB_HUMAN |
| | | beta | | | |
| 8491 | MLLADQGQSWKEEV | Glutathione S-transferase P | 20 | 33 | GSTP1_HUMAN |
| 8619 | AVHYLDETEQWEK | Complement C3 | 1024 | 1036 | CO3_HUMAN |
| 8727 | VGAHAGEYGAEALERM | Hemoglobin subunit alpha | 18 | 33 | HBA_HUMAN |
| 8919 | AHVDDmPNALSALSDL | Hemoglobin subunit alpha | 72 | 87 | HBA_HUMAN |
| 9151 | IAPQLSTEELVSLGEK | Afamin | 438 | 453 | AFAM_HUMAN |
| 9151 | ALWGKVNVDEVGGEALG | Hemoglobin subunit beta | 14 | 30 | HBB_HUMAN |
| 9380 | YAASSYLSLTPEQWK | Ig lambda-1 chain C regions | 194 | 208 | LAC1_HUMAN |
| 9554 | VANPSGNLTETYVQDR | Filamin-A | 1297 | 1312 | FLNA_HUMAN |
| 9602 | APVPTGEVYFADSFDR | Calnexin | 97 | 112 | CALX_HUMAN |
| 9956 | TALWGKVNVDEVGGEALG | Hemoglobin subunit beta | 13 | 30 | HBB_HUMAN |
| 10067 | SAVTALWGKVNVDEVGGE | Hemoglobin subunit beta | 10 | 27 | HBB_HUMAN |
| 10784 | FESFGDLSTPDAVmGNPK | Hemoglobin subunit beta | 43 | 60 | HBB_HUMAN |
| 12814 | SLDMDSIIAEVKAQYEDIAN | Keratin, type II cytoskeletal | 253 | 272 | K2C8_HUMAN |
| | | 8 | | | |
| 149779 | LSALSDLH | Hemoglobin subunit alpha | 81 | 88 | HBA_HUMAN |
| 149895 | GEALGRLLV | Hemoglobin subunit epsilon | 26 | 34 | HBE_HUMAN |
| 150158 | TPDAVMGNPKV | Hemoglobin subunit beta | 51 | 61 | HBB_HUMAN |
| 150166 | SASNMAIVDVK | Alpha-2-macroglobulin | 1374 | 1384 | A2MG_HUMAN |
| 150198 | ILDVLEEIPK | NADH dehydrogenase [ubiquinone] | 31 | 40 | NDUA5_HUMAN |
| | | 1 alpha subcomplex subunit 5 | | | |
| 150208 | TEVENGGSLGSK | Pyruvate kinase isozymes M1/M2 | 195 | 206 | KPYM_HUMAN |
| 150343 | NVDEVGGEALGRL | Hemoglobin subunit beta | 20 | 32 | HBB_HUMAN |
| 150355 | ISKQEYDESGPS | Actin, cytoplasmic 1 | 357 | 368 | ACTB_HUMAN |
| 150380 | EYVQLISVYEK | Olfactomedin-4 | 122 | 132 | OLFM4_HUMAN |
| 150403 | KVPQVSTPTLVEV | Serum albumin | 438 | 450 | ALBU_HUMAN |
| 150436 | AEFAEVSKLVTDL | Serum albumin | 250 | 262 | ALBU_HUMAN |
| 150453 | FFESFGDLSTPDA | Hemoglobin subunit beta | 42 | 54 | HBB_HUMAN |
| 150540 | DDmPNALSALSDLH | Hemoglobin subunit alpha | 75 | 88 | HBA_HUMAN |
| 150648 | LAKVDATEESDLAQQ | Protein disulfide-isomerase | 79 | 93 | PDIA1_HUMAN |
| 150697 | ImDPNIVGSEHYDVA | ATP synthase subunit beta, | 407 | 421 | ATPB_HUMAN |
| | | mitochondrial | | | |
| 150717 | SDPEQGVEVTGQYER | Inter-alpha-trypsin inhibitor | 745 | 759 | ITIH4_HUMAN |
| | | heavy chain H4 | | | |
| 150795 | GAFSDGLAHLDNLKGTF | Hemoglobin subunit beta | 70 | 86 | HBB_HUMAN |
| 29906 | LGPHAGDVEGHLS | Apolipoprotein A-IV | 329 | 341 | APOA4_HUMAN |
| 118597 | DGVSGGEGKGGSDGGGSHRK | CD99 antigen | 97 | 129 | CD99_HUMAN |
| | EGEEADAPGVIPG | | | | |
| 19773 | DFDDFNLED | CD99 antigen-like protein 2 | 26 | 34 | C99L2_HUMAN |
| 54687 | EpGSpGENGApGQMGPR | Collagen alpha-1(I) chain | 288 | 304 | CO1A1_HUMAN |
| 73697 | GNSGEpGApGSKGDTGAK- | Collagen alpha-1(I) chain | 431 | 453 | CO1A1_HUMAN |
| | GEPGp | | | | |
| 108327 | ERGSPGpAGPKGSpGEAGRp | Collagen alpha-1(I) chain | 510 | 539 | CO1A1_HUMAN |
| | GEAGLpGAKG | | | | |
| 46649 | SpGSPGPDGKTGPpGPAG | Collagen alpha-1(I) chain | 543 | 560 | CO1A1_HUMAN |
| 76839 | DGKTGpPGPAGQDGRPGPpG | Collagen alpha-1(I) chain | 550 | 572 | CO1A1_HUMAN |
| | ppG | | | | |
| 20334 | DGEAGAQGPpGPA | Collagen alpha-1(I) chain | 613 | 625 | CO1A1_HUMAN |
| 33727 | pPGEAGKpGEQGVP | Collagen alpha-1(I) chain | 651 | 664 | CO1A1_HUMAN |
| 23628 | KpGEQGVpGDLG | Collagen alpha-1(I) chain | 657 | 668 | CO1A1_HUMAN |
| 42304 | DGQpGAKGEpGDAGAK | Collagen alpha-1(I) chain | 820 | 835 | CO1A1_HUMAN |
| 28306 | RpGEVGPpGPpGP | Collagen alpha-1(I) chain | 918 | 930 | CO1A1_HUMAN |
| 24393 | GPpGESGREGApG | Collagen alpha-1(I) chain | 1007 | 1019 | CO1A1_HUMAN |
| 118224 | ESGREGAp- | Collagen alpha-1(I) chain | 1011 | 1042 | CO1A1_HUMAN |
| | GAEGSpGRDGSpGAKGDRGE | | | | |
| | TGPA | | | | |
| 74420 | EGSpGRDGSpGAKGDRGET- | Collagen alpha-1(I) chain | 1021 | 1042 | CO1A1_HUMAN |
| | GPA | | | | |
| 69769 | DGESGRPGRpGERGLpGPpG | Collagen alpha-1(III) chain | 230 | 249 | CO3A1_HUMAN |
| 70413 | DGESGRpGRpGERGLpGPpG | Collagen alpha-1(III) chain | 230 | 249 | CO3A1_HUMAN |
| 32470 | SpGGpGSDGKpGPpG | Collagen alpha-1(III) chain | 541 | 555 | CO3A1_HUMAN |
| 25866 | GPGGDKGDTGPpGP | Collagen alpha-1(III) chain | 624 | 637 | CO3A1_HUMAN |
| 156878 | LQGLpGTGGppGENGKpGEp | Collagen alpha-1(III) chain | 640 | 687 | CO3A1_HUMAN |
| | GpKGDAGAPGAPGGKGDA- | | | | |
| | GAPGERGppG | | | | |
| 61332 | ApGAPGGKGDAGAp- | Collagen alpha-1(III) chain | 667 | 687 | CO3A1_HUMAN |
| | GERGPpG | | | | |
| 60259 | ApGPQGPRGDkGETGERG | Collagen alpha-1(III) chain | 1084 | 1101 | CO3A1_HUMAN |
| 33973 | KGEAGLpGApGSPGQ | Collagen alpha-1(XIX) chain | 291 | 305 | COJA1_HUMAN |
| 78111 | GPpGKAGEDGHpGKPGRpGE | Collagen alpha-2(I) chain | 136 | 157 | CO1A2_HUMAN |
| | RG | | | | |
| 110841 | LkGQpGApGVKGEpGAp- | Collagen alpha-2(I) chain | 188 | 217 | CO1A2_HUMAN |
| | GENGTPGQTGARG | | | | |
| 112013 | LkGQpGApGVkGEpGAp- | Collagen alpha-2(I) chain | 188 | 217 | CO1A2_HUMAN |
| | GENGTpGQTGARG | | | | |
| 107360 | kGQpGApGVkGEpGAp- | Collagen alpha-2(I) chain | 189 | 217 | CO1A2_HUMAN |
| | GENGTpGQTGARG | | | | |
| 51804 | SpGNIGPAGKEGPVGLpG | Collagen alpha-2(I) chain | 455 | 472 | CO1A2_HUMAN |
| 75025 | DEAGSEADHEGTHSTKRGHA | Fibrinogen alpha chain | 605 | 624 | FIBA_HUMAN |
| 86426 | DEAGSEADHEGTHSTKRGHA | Fibrinogen alpha chain | 605 | 626 | FIBA_HUMAN |
| | KS | | | | |
| 98089 | DEAGSEADHEGTHSTKRGHA | Fibrinogen alpha chain | 605 | 628 | FIBA_HUMAN |
| | KSRP | | | | |
| 13746 | ATKTVGSDTF | Kininogen-1 | 62 | 71 | KNG1_HUMAN |
| 49958 | SGDSDDDEPPPLPRL | Membrane associated progester- | 54 | 68 | PGRC1_HUMAN |
| | | one receptor component 1 | | | |
| 111426 | IPVKQADSGSSEEKQLYNKY | Osteopontin | 17 | 42 | OSTP_HUMAN |
| | PDAVAT | | | | |
| 15800 | GEYKFQNAL | Serum albumin | 423 | 431 | ALBU_HUMAN |
| 48580 | TGLSMDGGGSPKGDVDP | Sodium/potassium-transporting | 2 | 18 | ATNG_HUMAN |
| | | ATPase subunit gamma | | | |
| 42404 | GLSMDGGGSPKGDVDP | Sodium/potassium-transporting | 3 | 18 | ATNG_HUMAN |
| | ATPase subunit | | | | |
| | gamma | | | | |
| 8503 | SGSVIDQSR | Uromodulin | 589 | 597 | UROM_HUMAN |
| 41514 | DQSRVLNLGPITR | Uromodulin | 594 | 606 | UROM_HUMAN |
| 65746 | SGSVIDQSRVLNLGPITR | Uromodulin | 589 | 606 | UROM_HUMAN |
| 72161 | SGSVIDQSRVLNLGPITRK | Uromodulin | 589 | 607 | UROM_HUMAN |
| 50212 | VGGGEQPPPAPAPRRE | Xylosyltransferase 1 | 51 | 66 | XYLT1_HUMAN |

For the evaluation of the measured presence or absence of the markers, especially the mass and CE time thereof, the reference values stated in Table 4 can be recurred to.

TABLE 4
Reference values for the evaluation of the
measured presence or absence of the markers.

| Protein/ | Mass | CE time |
|---|---|---|
| Polypeptide | [Da] | [min] |
| Aprotinin | 6513.09 | 19.3 |
| Ribonuclease | 13681.32 | 19.55 |
| Lysozyme | 14303.88 | 19.28 |
| REVQSKIGYGRQIIS | 1732.96 | 20.95 |
| ELMTGELPYSHINNRDQIIFMVGR | 2832.41 | 23.49 |
| TGSLPYSHIGSRDQIIFMVGR | 2333.19 | 22.62 |
| GIVLYELMTGELPYSHIN | 2048.03 | 32.2 |

For the evaluation of the measured amplitudes of the markers, the reference values stated in Table 5 can be recurred to.

TABLE 5
Reference values for the normalization of the marker amplitudes.

| Protein | Mass | CE time | | mean log amp |
|---|---|---|---|---|
| ID | [Da] | [min] | Frequency | (log median) |
| 137 | 830.47 | 31.75 | 0.59 | 2.19(2.14) |
| 457 | 871.53 | 24.67 | 0.7 | 2.59(2.68) |
| 1070 | 925.53 | 31.12 | 0.65 | 1.98(2.03) |
| 1536 | 958.56 | 24.46 | 0.8 | 2.96(3.07) |
| 1891 | 983.58 | 31.87 | 0.49 | 2.30(2.37) |
| 2409 | 1020.62 | 32.87 | 0.54 | 2.96(3.04) |
| 2459 | 1024.59 | 31.8 | 0.67 | 2.70(2.75) |
| 2710 | 1042.62 | 31.82 | 0.81 | 3.81(3.95) |
| 3102 | 1071.64 | 24.62 | 0.57 | 3.13(3.19) |
| 3144 | 1074.58 | 32.41 | 0.57 | 2.59(2.64) |
| 4216 | 1155.64 | 33.15 | 0.42 | 2.42(2.53) |
| 4332 | 1166.67 | 33.32 | 0.4 | 2.90(2.99) |
| 4531 | 1181.67 | 25.32 | 0.57 | 2.72(2.86) |
| 4613 | 1190.69 | 26.45 | 0.47 | 2.59(2.59) |
| 5318 | 1265.71 | 33.84 | 0.54 | 2.94(2.98) |
| 5556 | 1288.72 | 25.9 | 0.8 | 3.44(3.53) |
| 5688 | 1303.7 | 34.02 | 0.61 | 3.03(3.19) |
| 6183 | 1359.77 | 26.72 | 0.61 | 3.28(3.40) |
| 6353 | 1379.81 | 22.13 | 0.43 | 2.94(3.13) |
| 6539 | 1400.73 | 34.66 | 0.66 | 3.45(3.60) |
| 6888 | 1440.78 | 27.39 | 0.6 | 2.83(2.94) |
| 6954 | 1448.86 | 22.55 | 0.84 | 3.82(3.99) |
| 7636 | 1528.81 | 27.47 | 0.77 | 3.88(4.09) |
| 7948 | 1566.88 | 35.53 | 0.43 | 3.37(3.55) |
| 7964 | 1568.86 | 23.47 | 0.46 | 2.73(2.63) |
| 9722 | 1783.99 | 35.34 | 0.52 | 2.91(3.10) |
| 10093 | 1832.99 | 24.17 | 0.78 | 3.65(3.83) |
| 10111 | 1832.99 | 30.51 | 0.51 | 2.97(3.04) |
| 10683 | 1910.97 | 36.43 | 0.56 | 3.13(3.19) |
| 11033 | 1964.14 | 24.75 | 0.45 | 2.60(2.62) |
| 11562 | 2042.09 | 25.01 | 0.53 | 3.41(3.70) |
| 11587 | 2045.14 | 31.26 | 0.63 | 3.46(3.66) |
| 11950 | 2102.18 | 25.74 | 0.52 | 3.11(3.32) |
| 12447 | 2173.23 | 25.96 | 0.61 | 3.87(4.05) |
| 12726 | 2212.18 | 20.63 | 0.48 | 3.44(3.54) |
| 14383 | 2461.26 | 37.67 | 0.55 | 2.73(2.78) |
| 15154 | 2581.39 | 27.48 | 0.49 | 3.61(3.69) |
| 18320 | 3194.75 | 23.14 | 0.51 | 4.24(4.43) |

The evaluation of the polypeptides measured can be done on the basis of the presence or absence or amplitude of the markers taking the following limits into account:

Specificity is defined as the number of actually negative samples divided by the sum of the numbers of the actually negative and false positive samples. A specificity of 100% means that a test recognizes all healthy persons as being healthy, i.e., no healthy subject is identified as being ill. This says nothing about how reliably the test recognizes sick patients.

Sensitivity is defined as the number of actually positive samples divided by the sum of the numbers of the actually positive and false negative samples. A sensitivity of 100% means that the test recognizes all sick persons. This says nothing about how reliably the test recognizes healthy patients.

By the markers according to the invention, it is possible to achieve a specificity of at least 65%, preferably at least 75%, more preferably 85%, for the recognition of a bile duct stricture or a CCC.

By the markers according to the invention, it is possible to achieve a sensitivity of at least 65%, preferably at least 75%, more preferably 85%, for the recognition of a bile duct stricture or a CCC.

The migration time is determined by capillary electrophoresis (CE), for example, as set forth in the Example under item 2. Thus, a glass capillary of 90 cm in length and with an inner diameter (ID) of 50 μm and an outer diameter (OD) of 360 μm is operated at a voltage of 30 kV. As the solvent for the sample, 20% acetonitrile, 0.25% formic acid in water is used, for example.

It is known that the CE migration times may vary. Nevertheless, the order in which the polypeptide markers are eluted is typically the same for any CE system employed. In order to balance the differences in the migration time, the system may be normalized using standards for which the migration times are known. These standards may be, for example, the polypeptides stated in the Examples (see the Example, item 3). The variation of the CE times between individual measurements is relatively small, typically within a range of ±2 min, preferably within a range of ±1 min, more preferably ±0.5 min, even more preferably ±0.2 min or 0.1 min.

The characterization of the polypeptide markers shown in Tables 1 to 5 was determined by means of capillary electrophoresis-mass spectrometry (CE-MS), a method which has been described in detail, for example, by Neuhoff et al. (Rapid Communications in mass spectrometry, 2004, Vol. 20, pp. 149-156). The variation of the molecular masses between individual measurements or between different mass spectrometers is relatively small when the calibration is exact, typically within a range of ±0.1%, preferably within a range of ±0.05%, more preferably within a range of ±0.03%, even more preferably within a range of ±0.01% or 0.005%.

The polypeptide markers according to the invention are proteins or peptides or degradation products of proteins or peptides. They may be chemically modified, for example, by posttranslational modifications, such as glycosylation, phosphorylation, alkylation or disulfide bridges, or by other reactions, for example, within the scope of the degradation.

Proceeding from the parameters that determine the polypeptide markers (molecular weight and migration time), it is possible to identify the sequence of the corresponding polypeptides by methods known in the prior art.

The polypeptides according to the invention are used for the differentiation of benign and malignant bile duct strictures from choledocholithiasis, and for the differentiation of a CCC from a PSC in an unclear bile duct stenosis.

“Diagnosis” means the process of knowledge gaining by assigning symptoms or phenomena to a disease or injury. In the present case, the presence or absence of particular polypeptide markers is also used for differential diagnostics. The presence or absence of a polypeptide marker can be measured by any method known in the prior art. Methods which may be known are exemplified below.

A polypeptide marker is considered present if its measured value is at least as high as its threshold value. If the measured value is lower, then the polypeptide marker is considered absent. The threshold value can be determined either by the sensitivity of the measuring method (detection limit) or empirically.

In the context of the present invention, the threshold value is considered to be exceeded preferably if the measured value of the sample for a certain molecular mass is at least twice as high as that of a blank sample (for example, only buffer or solvent).

The polypeptide marker or markers is/are used in such a way that its/their presence or absence is measured, wherein the presence or absence is indicative of the diagnosis of a benign or malignant bile duct stricture or a CCC. Thus, there are polypeptide markers which are typically present in subjects with a benign or malignant bile duct stricture or in subjects with a CCC, but occur less frequently or are absent in subjects with no bile duct stricture, for example, those suffering from choledocholithiasis, or CCC, for example those with PSC. Further, there are polypeptide markers which are present in patients with bile duct stenoses of different origin, but are less frequently or not at all present in patients with PSC or CCC.

In addition or also alternatively to the frequency markers (determination of presence or absence), amplitude markers may also be used for diagnosis. Amplitude markers are used in such a way that the presence or absence is not critical, but the height of the signal (the amplitude) decides if the signal is present in both groups. Two normalization methods are possible to achieve comparability between differently concentrated samples or different measuring methods. In the first approach, all peptide signals of a sample are normalized to a total amplitude of 1 million counts. Therefore, the respective mean amplitudes of the individual markers are stated as parts per million (ppm).

In addition, it is possible to define further amplitude markers by an alternative normalization method: In this case, all peptide signals of one sample are scaled with a common normalization factor. Thus, a linear regression is formed between the peptide amplitudes of the individual samples and the reference values of all known polypeptides. The slope of the regression line just corresponds to the relative concentration and is used as a normalization factor for this sample.

The decision for a diagnosis is made as a function of how high the amplitude of the respective polypeptide markers in the patient sample is in comparison with the mean amplitudes in the control groups or the “ill” group. If the value is close to the mean amplitude of the “ill” group, the existence of a benign or malignant stricture and the absence of choledocholithiasis is to be considered in the polypeptide markers for recognizing a bile duct stricture, and the existence of a CCC in the presence of a PSC(CCC on top of PSC), but also the existence of a CCC in the absence of a PSC, is to be considered in the polypeptide markers for recognizing a CCC. However, if it rather corresponds to the mean amplitudes of the control group, the non-existence of a benign or malignant stricture is to be considered in the polypeptide markers for recognizing a benign or malignant bile duct stricture, and the non-existence of a CCC is to be considered in the polypeptide markers for recognizing a CCC. The distance between the measured value and the mean amplitude can be interpreted as a probability of the sample's belonging to a certain group.

Alternatively, the distance between the measured value and the mean amplitude may be considered a probability of the sample's belonging to a certain group.

A frequency marker is a variant of an amplitude marker in which the amplitude in some samples is so low that it is below the detection limit. It is possible to convert such frequency markers to amplitude markers by including the corresponding samples in which the marker is not found into the calculation of the amplitude with a very small amplitude, on the order of the detection limit.

The subject from which the sample in which the presence or absence of one or more polypeptide markers is determined is derived may be any subject which is capable of suffering from a benign or malignant bile duct stricture, or CCC. Preferably, the subject is a mammal, and most preferably, it is a human.

In a preferred embodiment of the invention, not just three polypeptide markers, but a larger combination of polypeptide markers are used. By comparing a plurality of polypeptide markers, a bias in the overall result from a few individual deviations from the typical presence probability in single individuals can be reduced or avoided.

The sample in which the presence or absence of the peptide marker or markers according to the invention is measured may be any sample which is obtained from the body of the subject. The sample is a sample which has a polypeptide composition suitable for providing information about the state of the subject. For example, it may be blood, urine, synovial fluid, a tissue fluid, a body secretion, sweat, cerebrospinal fluid, lymph, intestinal, gastric or pancreatic juice, bile, lacrimal fluid, a tissue sample, sperm, vaginal fluid or a feces sample. Preferably, it is a liquid sample.

In a preferred embodiment, the sample is a bile sample. In this case, the markers of Table 2b are suitable for diagnostics. If other samples are used, the markers of Table 2a are suitable. These markers are suitable if a urine sample is used as the sample, in particular. Urine samples are more readily obtained as compared to bile samples. However, bile samples seem to be of a higher significance.

In a preferred embodiment, the urine samples are used for diagnosis at first. Then, when the results are unclear, further analyses based on bile samples are performed.

Bile samples can be taken as known in the prior art. Preferably, a bile sample is taken in the course of an endoscopic intervention in the context of the present invention. For example, the bile sample may be taken from the bile duct by means of an endoscopically inserted catheter, or else by means of another apparatus.

The presence or absence of a polypeptide marker in the sample may be determined by any method known in the prior art that is suitable for measuring polypeptide markers. Such methods are known to the skilled person. In principle, the presence or absence of a polypeptide marker can be determined by direct methods, such as mass spectrometry, or indirect methods, for example, by means of ligands.

If required or desirable, the sample from the subject, for example, the urine sample, may be pretreated by any suitable means and, for example, purified or separated before the presence or absence of the polypeptide marker or markers is measured. The treatment may comprise, for example, purification, separation, dilution or concentration. The methods may be, for example, centrifugation, filtration, ultrafiltration, dialysis, precipitation or chromatographic methods, such as affinity separation or separation by means of ion-exchange chromatography, electrophoretic separation, i.e., separation by different migration behaviors of electrically charged particles in solution upon application of an electric field. Particular examples thereof are gel electrophoresis, two-dimensional polyacrylamide gel electrophoresis (2D-PAGE), capillary electrophoresis, metal affinity chromatography, immobilized metal affinity chromatography (IMAC), lectin-based affinity chromatography, liquid chromatography, high-performance liquid chromatography (HPLC), normal and reverse-phase HPLC, cation-exchange chromatography and selective binding to surfaces. All these methods are well known to the skilled person, and the skilled person will be able to select the method as a function of the sample employed and the method for determining the presence or absence of the polypeptide marker or markers.

In one embodiment of the invention, the sample, before being separated by capillary electrophoresis, is separated, purified by ultracentrifugation and/or divided by ultrafiltration into fractions which contain polypeptide markers of a particular molecular size.

Preferably, a mass-spectrometric method is used to determine the presence or absence of a polypeptide marker, wherein a purification or separation of the sample may be performed upstream from such method. As compared to the currently employed methods, mass-spectrometric analysis has the advantage that the concentration of many (>100) polypeptides of a sample can be determined by a single analysis. Any type of mass spectrometer may be employed. By means of mass spectrometry, it is possible to measure 10 fmol of a polypeptide marker, i.e., 0.1 ng of a 10 kD protein, as a matter of routine with a measuring accuracy of about ±0.01% in a complex mixture. In mass spectrometers, an ion-forming unit is coupled with a suitable analytic device. For example, electrospray-ionization (ESI) interfaces are mostly used to measure ions in liquid samples, whereas MALDI (matrix-assisted laser desorption/ionization) is used for measuring ions from a sample crystallized in a matrix. To analyze the ions formed, quadrupoles, ion traps or time-of-flight (TOF) analyzers may be used, for example.

In electrospray ionization (ESI), the molecules present in solution are atomized, inter alia, under the influence of high voltage (e.g., 1-8 kV), which forms charged droplets at first that become smaller from the evaporation of the solvent. Finally, so-called Coulomb explosions result in the formation of free ions, which can then be analyzed and detected.

In the analysis of the ions by means of TOF, a particular acceleration voltage is applied which confers an equal amount of kinetic energy to the ions. Thereafter, the time that the respective ions take to travel a particular drifting distance through the flying tube is measured very accurately. Since with equal amounts of kinetic energy, the velocity of the ions depends on their mass, the latter can thus be determined. TOF analyzers have a very high scanning speed and therefore reach a good resolution.

Preferred methods for the determination of the presence and absence of polypeptide markers include gas-phase ion spectrometry, such as laser desorption/ionization mass spectrometry, MALDI-TOF MS, SELDI-TOF MS (surface-enhanced laser desorption/ionization), LC MS (liquid chromatography/mass spectrometry), 2D-PAGE/MS and capillary electrophoresis-mass spectrometry (CE-MS). All the methods mentioned are known to the skilled person.

A particularly preferred method is CE-MS, in which capillary electrophoresis is coupled with mass spectrometry. This method has been described in some detail, for example, in the German Patent Application DE 10021737, in Kaiser et al. (J. Chromatogr A, 2003, Vol. 1013: 157-171, and Electrophoresis, 2004, 25: 2044-2055) and in Wittke et al. (J. Chromatogr. A, 2003, 1013: 173-181). The CE-MS technology allows to determine the presence of some hundreds of polypeptide markers of a sample simultaneously within a short time and in a small volume with high sensitivity. After a sample has been measured, a pattern of the measured polypeptide markers is prepared, and this pattern can be compared with reference patterns of a sick or healthy subjects. In most cases, it is sufficient to use a limited number of polypeptide markers for the diagnosis of UAS. A CE-MS method which includes CE coupled on-line to an ESI-TOF MS is further preferred.

For CE-MS, the use of volatile solvents is preferred, and it is best to work under essentially salt-free conditions. Examples of such solvents include acetonitrile, methanol and the like. The solvents can be diluted with water or admixed with an acid (e.g., 0.1% to 1% formic acid) in order to protonate the analyte, preferably the polypeptides.

By means of capillary electrophoresis, it is possible to separate molecules by their charge and size. Neutral particles will migrate at the speed of the electroosmotic flow upon application of a current, while cations are accelerated towards the cathode, and anions are delayed. The advantage of the capillaries in electrophoresis resides in the favorable ratio of surface to volume, which enables a good dissipation of the Joule heat generated during the current flow. This in turn allows high voltages (usually up to 30 kV) to be applied and thus a high separating performance and short times of analysis.

In capillary electrophoresis, silica glass capillaries having inner diameters of typically from 50 to 75 μm are usually employed. The lengths employed are, for example, 30-100 cm. In addition, the separating capillaries are usually made of plastic-coated silica glass. The capillaries may be either untreated, i.e., expose their hydrophilic groups on the interior surface, or coated on the interior surface. A hydrophobic coating may be used to improve the resolution. In addition to the voltage, a pressure may also be applied, which typically is within a range of from 0 to 1 psi. The pressure may also be applied only during the separation or altered meanwhile.

In a preferred method for measuring polypeptide markers, the markers of the sample are separated by capillary electrophoresis, then directly ionized and transferred on-line into a coupled mass spectrometer for detection.

In the method according to the invention, it is advantageous to use several polypeptide markers for diagnosis.

The use of at least 5, 6, 8 or 10 markers is preferred.

In one embodiment, 20 to 50 markers are used.

In order to determine the probability of the existence of a disease when several markers are used, statistic methods known to the skilled person may be used. For example, the Random Forests method described by Weissinger et al. (Kidney Int., 2004, 65: 2426-2434) may be used by using a computer program such as S-Plus, or the support vector machines as described in the same publication.

### EXAMPLE

### 1. Sample Preparation

For detecting the polypeptide markers for diagnosis, bile was employed. Bile was collected from patients with choledocholithiasis, from patients with PSC, from patients with CCC, and from patients with CCC on top of PSC.

For the subsequent CE-MS measurement, the lipids, which are contained in the bile in an elevated concentration, were precipitated by adding 1-butanol and diisopropyl ether, and all macromolecular bile components (>10 kDa) were separated off by ultrafiltration. Thus, 700 μl of bile was collected and pipetted to 700 μl of a 1-butanol/diisopropyl ether mixture (4:6, v/v). The sample was subsequently mixed on a vortex shaker until a homogeneous yellowish emulsion had formed. After centrifugation at 13,000 rpm and 4° C. for 10 min, 500 μl of the lower, aqueous phase was withdrawn for the subsequent ultrafiltration. Thus, the aqueous phase from the lipid removal was admixed with 500 μl of 8 M (w/v) urea solution and loaded on the UF filter (10 kDa MWCO, Sartorius, Gottingen, Germany). Subsequently, 1.0 ml of distilled water was added, and the ultrafiltration was performed at 3000 rpm in a centrifuge until 1.1 ml of ultrafiltrate was obtained. The 1.1 ml of filtrate obtained was then applied to a PD 10 column (GE Healthcare, Munich, Germany) and eluted with 2.5 ml of 0.01% NH₄OH, and lyophilized. For the CE-MS measurement, the polypeptides were then resuspended with 20 μl of water (HPLC grade, Merck).

### 2. CE-MS Measurement

The CE-MS measurements were performed with a capillary electrophoresis system from Beckman Coulter (P/ACE MDQ System; Beckman Coulter Inc., Fullerton, Calif., USA) and an ESI-TOF mass spectrometer from Bruker (micro-TOF MS, Bruker Daltonik, Bremen, Germany).

The CE capillaries were supplied by Beckman Coulter and had an ID/OD of 50/360 μm and a length of 90 cm. The mobile phase for the CE separation consisted of 20% acetonitrile and 0.25% formic acid in water. For the “sheath flow” on the MS, 30% isopropanol with 0.5% formic acid was used, here at a flow rate of 2 μl/min. The coupling of CE and MS was realized by a CE-ESI-MS Sprayer Kit (Agilent Technologies, Waldbronn, Germany).

For injecting the sample, a pressure of from 1 to a maximum of 6 psi was applied, and the duration of the injection was 99 seconds. With these parameters, about 150 nl of the sample was injected into the capillary, which corresponds to about 10% of the capillary volume. A stacking technique was used to concentrate the sample in the capillary. Thus, before the sample was injected, a 1 M NH₃ solution was injected for 7 seconds (at 1 psi), and after the sample was injected, a 2 M formic acid solution was injected for 5 seconds. When the separation voltage (30 kV) was applied, the analytes were automatically concentrated between these solutions.

The subsequent CE separation was performed with a pressure method: 40 minutes at 0 psi, then 0.1 psi for 2 min, 0.2 psi for 2 min, 0.3 psi for 2 min, 0.4 psi for 2 min, and finally 0.5 psi for 32 min. The total duration of a separation run was thus 80 minutes.

In order to obtain as good a signal intensity as possible on the side of the MS, the nebulizer gas was turned to the lowest possible value. The voltage applied to the spray needle for generating the electrospray was 3700-4100 V. The remaining settings at the mass spectrometer were optimized for peptide detection according to the manufacturer's instructions. The spectra were recorded over a mass range of m/z 400 to m/z 3000 and accumulated every 3 seconds.

### 3. Standards for the CE Measurement

For checking and standardizing the CE measurement, the proteins or polypeptides mentioned in Table 4 which are characterized by the stated CE migration times under the chosen conditions were employed:

In principle, it is known to the skilled person that slight variations of the migration times may occur in separations by capillary electrophoresis. However, under the conditions described, the order of migration will not change. For the skilled person who knows the stated masses and CE times, it is possible without difficulty to assign their own measurements to the polypeptide markers according to the invention. For example, he may proceed as follows: At first, he selects one of the polypeptides found in his measurement (peptide 1) and tries to find one or more identical masses within a time slot of the stated CE time (for example, ±5 min). If only one identical mass is found within this interval, the assignment is completed. If several matching masses are found, a decision about the assignment is still to be made. Thus, another peptide (peptide 2) from the measurement is selected, and it is tried to identify an appropriate polypeptide marker, again taking a corresponding time slot into account.

Again, if several markers can be found with a corresponding mass, the most probable assignment is that in which there is a substantially linear relationship between the shift for peptide 1 and that for peptide 2.

Depending on the complexity of the assignment problem, it suggests itself to the skilled person to optionally use further proteins from his sample for assignment, for example, ten proteins. Typically, the migration times are either extended or shortened by particular absolute values, or compressions or expansions of the whole course occur. However, comigrating peptides will also comigrate under such conditions.

In addition, the skilled person can make use of the migration patterns described by Zuerbig et al. in Electrophoresis 27 (2006), pp. 2111-2125. If he plots his measurement in the form of m/z versus migration time by means of a simple diagram (e.g., with MS Excel), the line patterns described also become visible. Now, a simple assignment of the individual polypeptides is possible by counting the lines.

Other approaches of assignment are also possible. Basically, the skilled person could also use the peptides mentioned above as internal standards for assigning his CE measurements.

1. A process for the differential diagnosis between a benign or malignant bile duct stricture and a choledocholithiasis, comprising the step of measuring the presence or absence or amplitude of at least three polypeptide markers in a sample of body fluid, wherein said polypeptide markers are selected from the markers characterized in Table 1 by values for the molecular masses and migration times.

2. The process according to claim 1, wherein an evaluation of the markers is effected by means of the reference values stated in Table 1.

3. A process for the differential diagnosis between a cholangiocellular carcinoma and primary sclerosing cholangitis, comprising the step of measuring the presence or absence or amplitude of at least three polypeptide markers in a sample of body fluid, wherein said polypeptide markers are selected from the markers characterized in Table 2a by values for the molecular masses and migration times if the sample is a non-bile sample, and said polypeptide markers are selected from the markers characterized in Table 2b by values for the molecular masses and migration times if the sample is a bile sample.

4. The process according to claim 3, wherein an evaluation of the markers is effected by means of the reference values stated in Tables 2a and 2b.

5. The process according to claim 1, wherein at least five, at least six, at least eight, at least ten, at least 20 or at least 50 polypeptide markers as defined in claim 1 are used.

6. The process according to claim 3, wherein a urine sample is used as said non-bile sample.

7. The process according to claim 1, wherein capillary electrophoresis, HPLC, gas-phase ion spectrometry and/or mass spectrometry is used for measuring said polypeptide markers.

8. The process according to claim 1, wherein a capillary electrophoresis is performed before the molecular mass of said polypeptide markers is measured.

9. The process according to claim 1, wherein mass spectrometry is used for measuring the presence or absence of said polypeptide markers.

10. (canceled)

11. (canceled)

12. A method for the differential diagnosis between a benign or malignant bile duct stricture and a choledocholithiasis, comprising the steps:
a) separating a sample into at least five, subsamples;
b) analyzing at least five subsamples for determining three polypeptide markers in the sample, wherein said polypeptide markers are selected from the markers of Table 1, which are characterized by their molecular masses and migrations times (CE times).

13. A method for the differential diagnosis between a cholangiocellular carcinoma and primary sclerosing cholangitis, comprising the steps:
a) separating a sample into at least five, subsamples;
b) analyzing at least five subsamples for determining three polypeptide markers in the sample, wherein said polypeptide markers are selected from the markers of Table 2a, which are characterized by their molecular masses and migrations times (CE times), if the sample is a non-bile sample, and said polypeptide markers are selected from the markers characterized by their molecular masses and migrations times in Table 2b if the sample is a bile sample.

14. The method according to claim 13, wherein at least 10 subsamples are separated and measured.

15. The method according to claim 13, wherein the CE time is based on a 90 cm length glass capillary having an inner diameter (ID) of 50 μm at an applied voltage of 25 kV, wherein 20% acetonitrile, 0.25 M formic acid in water is used as the mobile solvent.

16. The method according to claim 1, wherein the sensitivity is at least 60% and the specificity is at least 40%.

17. The method according to claim 3, wherein the sensitivity is at least 60% and the specificity is at least 40%.

18. The method according to claim 12, wherein the sensitivity is at least 60% and the specificity is at least 40%.

19. The method according to claim 13, wherein the sensitivity is at least 60% and the specificity is at least 40%.

20. The method according to claim 3 wherein capillary electrophoresis, HPLC, gas-phase ion spectrometry and/or mass spectrometry is used for measuring said polypeptide markers.

21. The method according to claim 3 wherein a capillary electrophoresis is performed before the molecular mass of said polypeptide markers is measured.

22. The method according to claim 3 wherein mass spectrometry is used for measuring the presence or absence of said polypeptide markers.
